SEQUENCE LISTING (3 sequences)

SEQ ID NO: 1 - protein, 12 aa, Artificial sequence
  Synthetic amino acid
CGPQGIWGQGCR

SEQ ID NO: 2 - protein, 12 aa, Artificial sequence
  Synthetic amino acid
CGPQGIAGQGCR

SEQ ID NO: 3 - protein, 12 aa, Artificial sequence
  Synthetic amino acid
CGPQGPAGQGCR

(19) United States
(12) Patent Application Publication
(10) Pub. No.: US 2022/0333050 A1
(43) Pub. Date: Oct. 20, 2022

(54) INTERFACIAL SEEDING OF CELLS AND PARTICLES ON SURFACES FOR DIAGNOSTICS AND THERAPEUTICS

(71) Applicant: William Marsh Rice University, Houston, TX (US)

(72) Inventors: Jordan MILLER, Houston, TX (US); Ian KINSTLINGER, Solon, OH (US); Bagrat GRIGORYAN, Spring, TX (US); Samantha PAULSEN, Newton, MA (US)

(73) Assignee: William Marsh Rice University, Houston, TX (US)

(21) Appl. No.: 17/716,389

(22) Filed: Apr. 8, 2022

Related U.S. Application Data
(60) Provisional application No. 63/173,118, filed on Apr. 9, 2021.

(51) Int. Cl. C12M 3/00; A61L 27/52; A61L 27/56; A61L 27/54; A61L 31/14

(52) U.S. Cl. CPC C12M 21/08; A61L 27/52; A61L 27/56; A61L 27/54; A61L 31/145; C12N 2533/54

(57) ABSTRACT

Some embodiments of the disclosure disclose a process for adhering cells, beads or particles to a surface of a material. The surface may be flat or curved and can be employed in the context of patterned or 3D printed hollow channels, facilitating the recapitulation of certain aspects of physiological systems. In various embodiments, interfacial cell seeding is accomplished by locally polymerizing a carrier containing a suspension of cells along the surface with a crosslinking molecule incorporated into the target material in advance of the interfacial polymerization. Polymerization of the carrier entraps the cells into controlled configuration along the surface. The techniques disclosed herein can be utilized for tissue engineering to build engineered organs/devices suitable for implanting into living organisms.

### PRIORITY CLAIM

This application claims benefit of priority to U.S. Provisional Application Ser. No. 63/173,118, filed Apr. 9, 2021, the entire contents of which are hereby incorporated by reference.

### STATEMENT REGARDING FEDERALLY SPONSORED RESEARCH

This invention was made with government support under Grant Nos. F31 HL140905 and F31 HL134295 awarded by the National Institutes of Health and Grant No. DGE-1450681 awarded by the National Science Foundation. The government has certain rights in the invention.

### FIELD OF THE DISCLOSURE

The present specification generally relates to the fields of biology, medicine, medical devices and transplantation, and more specifically, to processes for adhering cells, beads or particles to a surface of a material, allowing for the recapitulation of certain aspects of physiological systems, such as in implantable devices.

### BACKGROUND

Endothelial cells (ECs) represent the primary functional cell type in the mammalian vascular system. Structurally, these cells form the inner (lumenal) lining of arteries and veins and comprise nearly the entirety of capillaries. Functionally, ECs serve as a semi-permeable barrier between blood and tissues and regulate the transport of metabolites and other molecules between the bloodstream and organs such as lung, liver, and kidney. ECs also play a key dynamic role in adapting the vascular system to the needs of neighboring tissue; they can reorganize and form new blood vessels (e.g., in embryonic development or in wound healing) in response to signals from other cell types. In analogy to endothelial cells, epithelial cells comprise the inner cellular layer within the pulmonary airway network as well as several ductal networks across organ systems (e.g., pancreas and kidney). Other cell types including smooth muscle cells are arranged circumferentially along the surface of tubular structures in the vascular and lymphatic networks. The cell types described above are all natively found lining the lumen (i.e., inner space) of a vascular or other fluidic network in the body, referred collectively as lumenal cells (LCs).

### SUMMARY

Various embodiments of the present disclosure disclose a method, comprising: providing a target material having a surface; incubating the surface with a polymerizable material or a cross-linking agent, and a carrier composition including (i) the cross-linking agent or the polymerizable material, respectively, and (ii) a cell, a bead or a particle; and washing the surface to remove excess carrier composition. In various embodiments, the cell, the bead, or the particle is immobilized on or in an interfacial layer of the material polymerized on the surface.

Various embodiments of the present disclosure disclose a cell coated lumenal surface comprising: a lumenal surface; an interfacial layer of polymerized material disposed on said lumenal surface; and a cell embedded in said interfacial layer.

Various embodiments of the present disclosure disclose a method of coating a target material, the method comprising: providing a target material having a surface; incubating the surface with a carrier composition comprising a cell, a bead, or a particle, and a temperature- or pH-polymerizable material; and exposing the surface to a temperature or pH that catalyzes polymerization of said material. In various embodiments, the cell, the bead, or the particle is immobilized on or in an interfacial layer of the material polymerized on the surface.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are not intended to be drawn to scale. Like reference numbers and designations in the various drawings indicate like elements. For purposes of clarity, not every component may be labeled in every drawing. In the drawings:

FIG. 1 is an example schematic flowchart illustrating the seeding of luminal cells via interfacial gel polymerization, according to various embodiments.

FIG. 2 shows example illustration of rapid endothelialization of patterned vascular channels via interfacial gel polymerization in the presence of a cross-linking agent, according to various embodiments.

FIG. 3 shows example illustration of sparse seeding of vascular channels via interfacial gel polymerization in the absence of a cross-linking agent, according to various embodiments.

FIGS. 4-7B show example luminal cell morphogenesis over several days, i.e., at day 0 (FIG. 4), day 2 (FIG. 5), day 6 (FIG. 6), and day 11 (FIGS. 7A-7B), following seeding through interfacial polymerization, according to various embodiments.

FIG. 8 is a flowchart for a method of coating a target material using a cross-linking agent to catalyze polymerization of a carrier composition, according to various embodiments.

FIG. 9 is a flowchart for a method of coating a target material using pH or temperature to catalyze polymerization of a carrier composition, according to various embodiments.

### DETAILED DESCRIPTION

The crucial role of endothelial cells (ECs) in human physiology has made them a high-priority target for incorporation into engineered models of tissue and organ function. The relevance and need for lumenal cells (LCs) within engineered tissues has expanded dramatically with the emergence of new technologies to fabricate vascular networks in soft hydrogels, especially through 3D printing. These techniques permit the creation of hollow, perfusable networks of channels in hydrogels. To utilize such channels as functional models of mammalian blood vessels, airway channels, lymphatics, or other ductal systems, LCs are be seeded inside the channels such that they form a coating along the inner wall.

One approach to seeding LCs along the surface of a patterned vascular channel has been to inject a high-density suspension of LCs (in cell culture media) into the network. In this approach, cells settle onto the inner channel surface due to gravity and can adhere directly to the surface. The construct can be physically rotated during the seeding process to facilitate even coverage of the surface, allowing for successful seeding of patterned vascular channels with good coverage and characteristic endothelial cell morphology. However, this approach is quite time-consuming, requiring multiple hours of incubation to successfully adhere endothelial cells and, in the inventors' experiments, optimal cell adhesion was observed when the incubation times exceeded four hours. Moreover, the lengthy incubation step may require frequent intervention to rotate the construct as needed, or custom equipment to automate this process. The time-intensive nature of this process is limiting not only in terms of the duration of the experiment, but also in terms of how extensively this process can be scaled up. Thus, improved methodologies are needed to advance this area of research.

Accordingly, the inventors disclose herein a new approach to adhering cells, beads, particles, etc., along a surface, such as a lumenal surface, of materials such as hydrogels or biomaterials. Broadly, this method permits virtually any type of biological cell (including but not limited to endothelial cells, epithelial cells, smooth muscle cells, fibroblasts, pericytes, and/or the like), beads, particles, etc., to be disposed along surfaces—both flat and curved. When applied in the context of patterned or 3D printed hollow channels, this process allows for the rapid application of cells along the inner surface of a channel network, as is observed in blood vessels (endothelial cell layer) or the airways of the lungs (epithelial cells). Thus, the technique may be utilized to recapitulate certain aspects of physiological systems. Although the discussion herein about interfacial seeding relates to cells, it is equally applicable to the disposition of beads, particles, etc., on surfaces as well.

In various embodiments, interfacial cell seeding can be accomplished by locally polymerizing a carrier composition or material containing a suspension of cells along the surface of a target material (e.g., target hydrogel or biomaterial (i.e., the material onto which cells are to be seeded)). The carrier composition can be a liquid or a gas. Beads, particles, etc., can be also seeded on the surface using a similar process where the carrier composition contains a suspension of beads, particles, etc. The polymerization reaction can be mediated by a crosslinking molecule, which is incorporated into the target material in advance of the interfacial polymerization. Pre-incorporating the cross-linking agent (alternatively referred herein as “crosslinker”) into the target material is sufficient to restrict polymerization to the interface between the target material and the carrier composition. Polymerization of the carrier gel or solution entraps or immobilizes the cells, beads, particles, etc., into an initial configuration along the surface, after which the cells, beads, particles, etc., may migrate and interact with one another to further assemble structures of biological interest. In various embodiments, polymerization occurs via the formation of a covalent or non-covalent bonding. Polymerization may also be induced by light or pH. In various embodiments, the interfacial layer produced on the surface can be orthogonal or conformal to said surface.

An important advantage of interfacial polymerization is the freedom to attach cells, beads, particles, etc., to surfaces with a wide range of topologies. Because cell seeding through interfacial polymerization operates as a conformal coating process (that is, the applied coating follows the curvature of the underlying substrate), it may be used upon target surfaces even with exotic or irregular curvatures. Such surface topologies are a hallmark of both biological systems as well as engineered materials which are designed to mimic native physiology. For example, in biological tissues and medical devices, it is common for fluids (such as blood, bile, or urine) to be transported through cylindrical channels. Within an organ (such as the liver or kidney), there may exist multiple independent networks of such cylindrical channels arranged in a hierarchical tree-like structure. In the lung, gases are exchanged between air and blood in balloon-like sacs (alveoli) with irregular curved surfaces. In spite of the variegated and complex surfaces presented in the above examples, interfacial seeding may be applied to adhere cells, beads, particles, etc., along any of these types of surfaces, in addition to more elementary surfaces such as a flat sheet of material, or a flat surface modified with grooves or ridges. As such, applications encompassing the adhesion of endothelial cells along the interior surface of a vascular tree, or the adhesion of epithelial cells along the inner surface of a lung airway, are expected to simultaneously be facilitated by the disclosed interfacial seeding techniques.

As discussed below, various embodiments of interfacial seeding utilize a cross-linking agent (i.e., crosslinker) which diffuses from the target material into the carrier composition. In these embodiments, the thickness or depth of the interfacially polymerized carrier composition is related to the concentration of the crosslinker, as well as the duration of the polymerization reaction. Therefore, the thickness can be controlled by modulating the crosslinking time and initial crosslinker concentration to yield interfacial layers spanning over an order of magnitude in thickness. In various embodiments, it may be desirable to adjust the thickness of the interfacially polymerized layer depending such that the thickness matches the size of the adhered cells, beads, particles, etc.

Moreover, interfacial cell/beads/particles seeding with a diffusible crosslinker is not limited to a single polymerization step. Rather, multiple polymerizations can be executed successively to yield multi-layered structures (that is, concentric layers of interfacially polymerized cells or particles). Such embodiments are expected to be important for developing engineered devices with concentric cell layers, such as vascular networks with an endothelial layer atop a smooth muscle layer. For example, the surface on which interfacial seeding occurs may be located adjacent to either of smooth muscle cells or endothelial cells of concentric layers of the smooth muscle cells and the endothelial cells. As another example, the surface is located in between the concentric layers of the smooth muscle cells and the endothelial cells. Further embodiments could include a layer of sensor particles or drug delivering particles alongside a layer of cells.

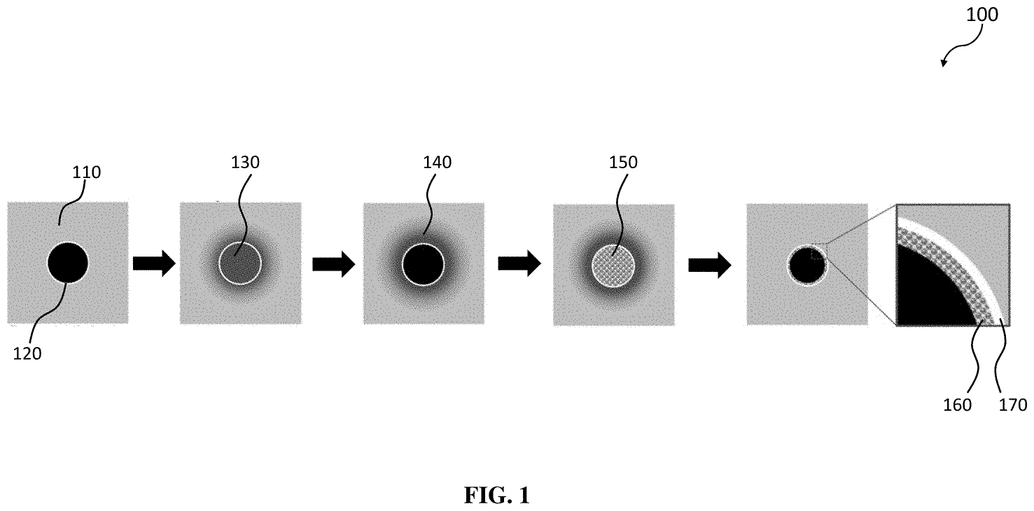

FIG. 1 is an example schematic flowchart illustrating the seeding of luminal cells via interfacial gel polymerization, according to various embodiments. In various embodiments, as an alternative to seeding ECs directly onto the surfaces of patterned channels, spatially controlled hydrogel polymerization is employed to entrap or immobilize ECs in a thin layer of a gel along the channel surface. In various embodiments, a target material 110 having an open channel 120 with a surface is provided. In various embodiments, the surface can be an outside of a tube having one or more openings. In various embodiments, the surface can be a cylindrical channel, a hemicylinder, an open void, or a cavity including one or more openings. In various instances, the target material can be a hydrogel. For example, the hydrogel can be a 3D-printed hydrogel (e.g., 3D-printed by stereolithography), or formed by casting around a sacrificial template including a vascular template. For instance, the sacrificial template can be made of a carbohydrate-based material formed through extrusion or selective laser sintering. In various embodiments, the target material can be a biomaterial, including but not limited to fibrin, gelatin, hyaluronic acid, agarose, alginate, collagen, or decellularized extracellular matrix. In various embodiments, the target material can be decellularized tissue or organ, including but not limited to artery, vein, lymphatic vessel, trachea, esophagus, lung, liver, kidney, pancreas, ureter, bladder, intestines, urethra, and/or the like.

In various embodiments, the target material 110 can be produced using a 3D printing process developed by the inventors that can fabricate 3D engineered tissues with biologically-inspired design criteria including, but not limited to, conforming to Murray's Law, multiscale branched vessels from tens to hundreds of micrometers in diameter, smooth inner walls, circular cross sections, and multiple inlet/outlets. Indeed, with printing parameter optimization, the limit to what can be fabricated depends on what one can model. Additionally, utilization of fractal space-filling models to computationally grow vascular networks around and through pre-existing vascular networks or following the architecture of native tissues can be achieved by computer growth models for even more complex and physiologically relevant 3D models. These mathematical fractal, space-filling models can be derived from, for example, knot theory, the Hilbert curve, and the L-system. Such mathematical fractal space-filling models to predict idealized vascular networks include, but are not limited to knot theory, Plumber's Nightmare, Peano curve, Hilbert curve, Pythagoras tree, and Brownian tree models. As an example, the Plumber's Nightmare model essentially comprises two Vascular Ladder models that are connected to each other by straight vertical cylinders. Multiple Plumber's Nightmare models can be intercalated such that they are interpenetrating. The Vascular Ladder models are comprised of 1 inlet and 1 outlet with two horizontal cylinders that are connected by diagonal cylinders, resulting in interchannel junctions.

Photopolymerizable hydrogel materials such as poly(ethylene glycol) diacrylate (PEGDA) can be crosslinked using a photoinitiator system such as lithium acylphosphinate (LAP) which absorbs in the UV to visible light wavelength range. By adding, for example, low concentrations of carbon black (which can absorb light across all UV-visible light spectrum), or low concentrations of tartrazine (which has a peak light absorption near 427 nm), the inventors can limit the depth of penetration of light. Other materials include—ene modified natural and synthetic materials that can be photopolymerized such as alginate, silk, dextran, chondroitin sulfate, hyaluronic acid, cellulose, heparin, and poly(caprolactone) and multi-component versions of these.

To achieve complex patterning of multilayered hydrogels, on the order of several centimeters, with high pattern fidelity, light exposure during the printing process is controlled so that the light projected onto the build platform interacts mainly with the layer that undergoes gelation for either partial or complete gelation. Radical mediated photopolymerization of hydrogels utilizes a photoinitiator—a molecule sensitive to a particular wavelength range that, upon light absorption, the molecule decays and release free radicals which can catalyze hydrogel polymerization. To this end, it is imperative to quantify the wavelength sensitivity of the photoinitiator. High concentrations of photoinitiator will absorb more light and provide higher z-resolution by limiting penetration depth of the incident light. However, high photoinitiator concentrations disrupt the photopolymerization reaction (more free radicals have a higher chance of annihilating each other), and photoinitiators at high concentrations are cytotoxic. In addition, with high x-y resolution from the projector, a complication is that light shines through the z-direction of the previously printed layers, potentially limiting the ability to form complex overhang structures (such as found in vasculature), and also may cause phototoxicity to entrapped cells.

Thus, to achieve high resolution printing, the present disclosure provides a photochemical means to provide, for the first time, high z-resolution in bioprinted tissues while maintaining high cell viability. To address the concerns outlined above, the inventors have identified a general strategy whereby biocompatible materials or chemicals are added to the pre-polymerization solution to provide higher z-resolution. The additive material is selected based on three criteria: 1) ability to absorb light wavelengths which fully encompass the photosensitive wavelength range of the photoinitiator, 2) limited participation or limited inhibition of photopolymerization reactions, and 3) biocompatibility at the concentrations desired. This additive material is referred to herein as a biocompatible, light-absorbing additive material suitable to control light penetration. Multiple molecules have been screened that absorb light, limiting the penetration depth of light into already formed layers. Suitable molecules absorb in the same region as the photoinitiator used in the pre-polymerization solution. Examples of molecules capable of controlling light penetration and therefore suitable for use as the biocompatible, light-absorbing additive material include carbon black, yellow food coloring, tartrazine, nanoparticles, microparticles, gold nanoparticles, riboflavin, phenol red, Beta-carotene, curcumin, saffron, and turmeric. Proteins may also act as suitable biocompatible, light-absorbing additive materials provided that their peak absorption overlaps with the peak absorption of the photoinitiator and matched to the incident light source. Additionally, the inventors recognize that cells that are transfected or transduced with proteins that absorb in the same region as the photoinitiator, such as cyan fluorescent protein (CFP) or green fluorescent protein (GFP), can be used at high concentrations, with reduced or no additives, to result in reduced lateral overcuring due to the light absorbing molecules present inside cells. Additionally, the inventors' methodology allows the printing of hydrogels with both horizontal and vertical channels due to stringent control of the penetration of the projected light.

Branching multi-scale transport systems are found in all multicellular life. Similar to the highly complex branching structure of vascular networks, the respiratory tree is also composed of a complex branching structure for sufficient supply of air in the distal regions of the lung. It has been indicated that endothelial cells may aid in lung epithelial branching. However, current manufacturing techniques do not allow for structures that mimic the anatomical complexity of native lung tissue. By using 3D printing, it should be possible to produce structures that mimic the anatomical complexity of native lung tissue and vasculature. The inventor's proposed approach allows for the printing of such structures and for embedding endothelial and epithelial cell types in channel lumens to mimic vascular and respiratory networks. The circular cross-sections that are attainable permit the development of confluent cell layers along the channel lumens. Given the higher z-resolution under the proposed approach, the channels can more closely mimic vascular and respiratory networks. The disclosed methods and materials facilitate the fine control of the geometry and architecture of multiple networks. By using fractal, space-filling models akin to physiological vascular networks, the technology permits the design and fabrication of relevant 3D constructs with interpenetrating channels.

Additionally, the proposed approach can be combined with other scaffold fabrication techniques, such as porogen leaching or surface coating, to result in physiologically relevant complex constructs with modified internal microarchitecture or surface properties. Additionally, the proposed approach can be used for fabrication of microfluidic devices for organ-on-a-chip or human-on-a-chip applications. Additionally, the printer can be modified to include specific sensors for ensuring printing of more precise layer thickness.

In various embodiments, the target material 110 can be a hydrogel matrix. The hydrogel matrix can include a first tubular channel and a second tubular channel. The hydrogel matrix can be porous. The hydrogel matrix can also include a first cell type and a second cell type embedded therein. In certain aspects, the hydrogel matrix can include a first cell type embedded therein. The hydrogel matrix can be produced in one or more layers and in certain embodiments, can include more than 1,000 layers, from about 10 layers to about 2,000 layers, from about 10 layers to about 1,000 layers, from about 10 layers to about 500 layers, from about 10 layers to about 100 layers, from about 100 layers to about 2,000 layers, from about 100 layers to about 1,000 layers, from about 100 layers to 500 layers, from about 100 layers to about 300 layers, from about 500 layers to about 1,000 layers, from about 500 layers to about 2,000 layers, from about 1,000 layers to about 2,000 layers, including values and subranges therebetween. In certain embodiments, one or more of the layers can have a thickness in the range from about 10 microns to about 100 microns, from about 50 microns to about 100 microns, from about 10 microns to about 50 microns, including values and subranges therebetween. In certain other aspects, one or more of the layers can have a thickness of about 50 microns, a thickness of less than about 50 microns, a thickness of about 25 microns, a thickness of about 100 microns, including values and subranges therebetween. In certain aspects, the one or more layers of the hydrogel matrix can include a first cell type wherein one or more other layers of the hydrogel matrix include a second cell type, but not the first cell type.

In various embodiments, the one or more layers of the hydrogel matrix can have cells embedded therein. In certain aspects, one or more layers of the hydrogel matrix adjacent to the one or more layers of the hydrogel matrix with embedded cells comprises an extracellular matrix protein. In other embodiments, the hydrogel matrix can be produced without layers. For example, the hydrogel matrix can be produced without layers using techniques including but not limited to needle casting, computed axial lithography, or xolography.

In various embodiments, the target material 110 can be a hydrogel matrix formed by casting around a sacrificial template including a vascular template. The inventors have also developed methods of generating perforated template structures that can be used as a scaffold for 3D printing approaches. The perforated structures may take the form of 3D dendritic carbohydrate lattices that may be used to cast vascularized engineered tissues. Furthermore, some embodiments are directed toward a process and composition of matter that may enable the fabrication of engineered vascular networks which are not constrained by the limitations of extrusion printing techniques described above.

Broadly, the methods for forming a perforated structure may include the steps of: solidifying a powder system by sintering or melting with an energy beam to form a three-dimensional structure to be used as a template; surface smoothing the template with a smoothing solution; surface coating the template with a surface coating material; backfilling a void space of the template with a matrix material; crosslinking the matrix material; and removing the template to form the perforated structure having channels shaped like the template. This method has been termed Selectively Laser Sintered-Carbohydrate Sacrificial Templating (SLS-CaST). Furthermore, they contemplate methods for computationally generating dendritic vascular networks called Mutual Tree Attraction. SLS of carbohydrates as described herein offers significant improvement in resolution, structural complexity, reproducibility, and throughput over previous methods of fabricating carbohydrate structures.

The appearance and quality of a three-dimensional structure formed via SLS may be influenced by various factors, including laser power density, laser scanning speed, and/or powder layer height. Proper control of these settings, according to one or more embodiments, may allow for consistent sintering of powders to create the three-dimensional structure. Improper values (or combinations of values) of factors such as these may result in the final geometry differing from an intended three-dimensional structure by lowering the resolution of features, adding unintended features, subtracting intended features, failing to fully fuse powder, creating balling defects, distorting the final three-dimensional structure, adding cavities, and/or other undesired alterations to the intended final geometry.

Laser power density and laser scanning speed may be interrelated. For instance, a low-powered laser moving with a very slow scanning speed may still impart excessive power to a region. Excessive power may cause over-sintering, distortions, cavities, and/or other undesired alterations to the intended final geometry. Over-sintering is when particles lying outside of the intended pattern are fused along with particles within the intended pattern being fused to form the intended final geometry. Over-sintering may occur in the z-axis (i.e., the build axis) and/or the x-axis/y-axis (i.e., the planar axes). In some embodiments, over-sintering may cause excessive fusion between successive powder layers, which may lower the resolution of the final geometry along the build axis and/or may add unintentional features in the build and/or planar axes. When the laser power density is too high and/or the laser scanning speed is too low, undesired alterations to the final geometry like over-sintering may occur. Additionally, low laser scanning speed may cause an irregular melt pool while the powder is sintered. The irregular melt pool may lead to additional undesired alterations to the intended final geometry, such as distortions and/or cavities. Alternatively, when the laser power density is too low and/or the laser scan speed is too fast, the powder may fail to form a continuously fused final three-dimensional structure. These circumstances may, in some embodiments, cause the balling defect that may sometimes be seen in SLS: when insufficiently melted, some powder may ball up into disconnected spheres instead of forming the final three-dimensional structure.

In various embodiments, the laser power density may be between in the range from between about 40 W/mm² to about 60 W/mm², from between about 40 W/mm² to about 55 W/mm², from between about 45 W/mm² to about 55 W/mm², from between about 40 W/mm² to about 50 W/mm², from between about 50 W/mm² to about 60 W/mm², including values and subranges therebetween. In various embodiments, the laser scanning speed may be in the range from about 1000 mm/min to about 2000 mm/min, from about 1250 mm/min to about 2000 mm/min, from about 1250 mm/min to about 1750 mm/min, from about 1000 mm/min to about 1750 mm/min, from about 1250 mm/min to about 2000 mm/min, from about 1500 mm/min to about 2000 mm/min, from about 1000 mm/min to about 1500 mm/min, including values and subranges therebetween.

In various embodiments, a powder system to be sintered into a structural material in the form of a three-dimensional structure may include one or more carbohydrate powders. The carbohydrate powders may include or consist of, for example: photoresist, agarose, gelatin, carbohydrates, sucrose, glucose, fructose, lactose, isomalt, dextran, cellulose, methylcellulose, poly(lactic acid), and/or poly(ethylene glycol). In one or more embodiments, the powder system may include one carbohydrate powder or a mixture of two or more carbohydrate powders. The powder system may be in powder form which includes a large number of powder granules. Isomalt, a sugar alcohol frequently used as an artificial sugar substitute, is one carbohydrate found to be compatible with SLS that may be sintered into three-dimensional structures such as vascular architectures, according to some embodiments. In some embodiments, the powder system may include one or both of isomalt and dextran powders.

The addition of an anti-caking agent may effectively augment powder flow while preserving sintering quality. Thus, according to some embodiments, the powder system for SLS may be a mixture of one or more carbohydrate powder(s) and one or more anticaking agent(s). In some embodiments, the anticaking agent may include one or more of: cornstarch, silicon dioxide, or xanthan gum or a mixture of two or more anti-caking agents. A three-dimensional structure formed from a powder system containing an anti-caking agent may include both the anti-caking agent and the carbohydrate powders in the final structure. Put another way, energy beam irradiation as occurs during SLS of such a powder system may sinter and/or melt both the carbohydrate powder(s) and the anti-caking agent(s) during solidification into the final three-dimensional structure.

In some embodiments, a three-dimensional structure formed of a structural material may take the form of a filament network that may be formed of a plurality of filaments, a three-dimensionally branched pattern, an interpenetrating geometry, and/or an unsupported geometry. The materials and/or methods, according to one or more embodiments of this disclosure, may be applied to fabricate a three-dimensional structure and thus a final geometry that may include various freeform structures and/or patterned fluidic networks. In some embodiments, such patterned fluidic networks serve as the substrate material for interfacial seeding of cells and/or particles. That is, the target material 110 can be formed as these patterned fluidic networks.

In various embodiments, the one or more layers of the hydrogel matrix that is the target material 110 can be formed from a photosensitive polymer. In certain aspects, the one or more layers of the hydrogel matrix can be formed from a second photosensitive polymer. The one or more layers of the hydrogel matrix can each include a first portion and second portion. In certain aspects, the first portion is formed from the photosensitive polymer and the second portion is formed from a second photosensitive polymer having a molecular weight of greater than about 2,000 Daltons. In certain aspects, the first portion can include a first cell type embedded therein and the second portion can include a second cell type embedded therein, wherein the first cell type is different from the second cell type. In certain aspects, the first portion can include a first fluorophore and the second portion can include a second fluorophore, wherein the first fluorophore is different from the second fluorophore.

In various embodiments, the hydrogel (e.g., target material 110) can include a first tubular channel and a second tubular channel. In certain aspects, the first and second tubular channel each can include a horizontal segment that intersects more than one layer of the bulk hydrogel matrix. The second tubular channel can interpenetrate the first channel where interpenetrating is defined as the spatial relationship between two channels wherein one channel intersects at least once a plane between two separate portions of the other channel. The tubular channels can also be branched. For example, the tubular channels may branch, as observed in the torus knot model, wherein the tubular channels reconverge at another point within the hydrogel. However, branched structures can also include channels which extend from the first tubular channel and/or the second tubular channel and terminate within the hydrogel. For, example, tree-like structures can be designed and produced using the present approach. In certain embodiments, the tubular channels have a diameter of 300 microns to 500 microns, 500 microns or less, 400 microns or less, 300 microns or less, including values and subranges therebetween. The tubular channel can also be perfu sable. In addition, the tubular channels can also be expandable in response to increases in pressure therein. Tubular channels can be lined with cells, including epithelial and endothelial cells. In certain aspects, the first tubular channel is lined with endothelial cells. In certain aspects, the second tubular channel is lined with epithelial cells.

In various embodiments, the first tubular channel can also include a first tubular inlet and a first tubular outlet on the surface of the hydrogel matrix. The second tubular channel can also include a second tubular inlet and a second tubular outlet on the surface of the hydrogel matrix. The first tubular channel can include a valve or other positive feature. Tubular channels can also include spikes that extend therefrom into the hydrogel matrix. Tubular channels can be filled with any appropriate fluid or gas. Such fluids or gases can include, by way of example but not limitation, bodily fluids and oxygen. In certain aspects, the first tubular channel can be filled with a fluid. In certain other aspects, the first tubular channel can be filled with culture media, red blood cells, blood, urine, bile and/or gases such as nitrogen and/or oxygen. In certain aspects, the second tubular channel can be filled with culture media, red blood cells, blood, urine, bile and/or gases such as nitrogen and/or oxygen. Tubular channels can also be filled with one or more different fluids and/or gases.

In various embodiments, the hydrogels of target material 110 can include more than two tubular channels. For example, target material 110 can include a third tubular channel and a fourth tubular channel. A tubular channel can interpenetrate more than one other tubular channel. For instance, a third tubular channel can interpenetrate a fourth tubular channel. Similarly, a second tubular channel can interpenetrate a first tubular channel and a third tubular channel. Tubular channel networks comprising multiple tubular channels may also interpenetrate at least one tubular channel or at least one other tubular channel network. For example, a third tubular channel may interpenetrate a first tubular channel that is also interpenetrated by a second tubular channel. As another example, a third tubular channel and fourth tubular channel can be interpenetrating and interpenetrate a first tubular channel or an interpenetrating network comprising a first tubular channel and a second tubular channel. In this manner, complex models can be constructed which permit complex interactions between tubular channels and tubular channel networks. The foregoing examples of multiple tubular channels are for exemplary purposes only and not intended to limit this disclosure.

It should be noted that list of polymers for various features of the present devices (e.g., target material 110) may overlap, as potential modifications to any given polymer can render it useful for the degradable or non-degradable portion. The degradable portion serves many purposes, including containing pro-angiogenic compounds, oxygen-releasing compounds, immune-modulating compounds, or other biologically active compounds, as well as cells, including endothelial cells. The degradable can affect the local site where it is implanted, including but not limited to vascularization, immunomodulation, and controlled release of other compounds, and/or confer useful advantages to the nondegradable portion of the device through these and other related means.

In various embodiments, target material 110 can be produced using a process for manufacturing a multi-layer hydrogel matrix construct. First, a 3D model of the multi-layer hydrogel matrix construct or target material 110 is created using a design software, wherein the 3D model of the multi-layer hydrogel matrix construct comprises a first computational algorithm that yields a first elongated void in the multilayer hydrogel matrix construct providing a first tubular channel, and a second computational algorithm that yields a second elongated void in the multi-layer hydrogel matrix construct providing a second tubular channel, wherein the second computational algorithm results in the second tubular channel interpenetrating the first tubular channel. The 3D model is then converted to a format suitable for use in a 3D printing software to yield a formatted 3D model. The formatted 3D model is then imported into the 3D printing software, wherein the 3D printing software is programmed to slice the 3D model into multiple two-dimensional (2D) xy images. A pre-polymerization solution is supplied to a vat associated with a build platform of a 3D printer, wherein the vat is transparent, and wherein the pre-polymerization solution comprises a photosensitive polymer having a molecular weight of greater than about 2,000 Daltons and at least two vinyl groups per molecule of polymer, a light-absorbing additive material to control light penetration, and a photoinitiator. The vat can also include a coating to which the hydrogel will not adhere such as a hydrophobic coating. For example, the coating can be polydimethylsiloxane (PDMS). This allows the hydrogel to separate from the vat without sticking. A mobile Z-axis stage of the 3D printer is positioned at a distance from the vat, wherein the Z-axis stage includes a surface sufficient for gelled material to adhere thereto, wherein the distance between the surface and an inner bottom surface of the vat is equivalent to a desired layer thickness of the tissue construct. A pattern is then projected on the inner bottom surface of the vat. For example, a light source may be projected through an optical configuration such as a digital light processing (DLP) system, to produce the pattern. A layer of the multi-layer hydrogel matrix construct is then polymerized. The steps of supplying a pre-polymerization solution, positioning the mobile Z-axis stage, projecting the light source and polymerizing a layer can be repeated one or more times, wherein the mobile Z-axis stage is moved so that the distance moved is equivalent to the desired thickness of each subsequent layer, and wherein the same or a different pattern is displayed for each subsequent layer. In certain aspects, at least the steps of supplying a pre-polymerization solution to a vat, positioning a mobile Z-axis stage, projecting a light source through an optical configuration, and polymerizing a layer of the multi-layer tissue construct are performed under hypothermic conditions. The hypothermic conditions can include a temperature of about 4° C. The optical configuration can include a collimator, a condenser, filters, and a DMD array. For example, the optical configuration can be part of a digital light processing system.

In various embodiments, the supplying, positioning, projecting, and polymerizing steps are performed at least once to produce a second layer, wherein the pre-polymerization solution used for the second layer comprises a second photosensitive polymer that is different from the photosensitive polymer used to fabricate the first layer. In certain aspects, the second photosensitive polymer can have a different molecular weight from the photosensitive polymer. In various embodiments, said steps can be performed a number of times sufficient to yield the multi-layered hydrogel matrix construct having the first tubular channel and the second tubular channel.

In certain embodiments, the first computational algorithm can be derived from knot theory. In certain aspects, the first computational algorithm and/or the second computational algorithm can be a Hilbert curve. The first computational algorithm and second computational algorithm can conform to Murray's Law. In certain aspects, the first computational algorithm can result in the first tubular channel being branched. The first computational algorithm and the second computational algorithm can include a mathematical space-filling model. The mathematical space-filling model can include the Plumber's Nightmare, Peano curve, Hilbert curve, Pythagoras tree, and Brownian tree models.

In various embodiments, the assembled 3D printer is an automated, computer-aided 3D prototyping device which utilizes additive manufacturing to selectively pattern photosensitive biomaterials one layer at a time, yielding a 3D tissue engineered construct. The printer contains a mobile Z-axis stage that is lowered onto the build platform which contains a vat with the pre-polymerization solution containing photosensitive polymers and a photoinitiator. Attached to the Z-axis stage is a surface onto which the gelled material adheres to. Additionally, the base of the printer houses a 45° mirror which reflects a horizontal projection onto the inner bottom surface of the transparent vat. The assembled printer also houses microelectronics that automate the printing process.

In order to print structures (e.g., target material 110), a 3D model is created in computer aided design (CAD) software and then exported to stereolithography (.stl) format. The .stl file is imported into software in which printing parameters are input. Then, the software computationally slices the 3D model into two-dimensional (2D) xy images, which act as dynamic photomasks, and uses the inputted print parameters to create commands for controlled automated printing. Once the transparent vat is filled with pre-polymerization mixture, the Z-axis stage is lowered onto the vat so that the distance between the surface and the inner bottom surface of the vat is the desired layer thickness. Then, a light source projects light through an optical setup containing a combination of collimator, condenser, filters such as dichroic or bandpass to select specific wavelengths of interest, and a digital micromirror device (DMD) array, such as a commercially available projector or a pico-projector, resulting in a pattern (the first layer of the 3D model), on the inner bottom surface of the vat, yielding a specific 3D patterned layer. After the material undergoes gelation, the Z-axis stage automatically moves up to the next layer height and the process is automatically continues with successive, automated projection/Z-axis stage movement until the final 3D construct is obtained.

Additionally, the power output from projector devices may not be homogenous. In such cases, the power output may be harmonized or flat-field, so that the projected pattern does not have unintentional heterogenous properties. To this end, a blank exposure may be projected onto a film of phosphors to obtain a luminescent image. This image contains a 2D matrix of intensity values that are essentially normalized and inverted in numerical computing programs, such as MATLAB. The inverted image essentially acts as a filter, resulting in a more homogenous power output to ensure that the gelation of materials is homogenous throughout the whole projected layer.

In various embodiments, modifications to this assembly can involve addition of a syringe setup to automatically dispense more pre-polymerization solution during the printing process, as necessary. To achieve more heterogenous constructs with different materials, a modification to the assembly involves modifying the build platform, vat, and/or the Z-axis stage so that multiple materials can be automatically printed with this technique. For example, the build platform can be designed in such a way as to house multiple vats with different materials for printing 3D hydrogels with multiple materials.

In various embodiments, the target material 110 can be a multiscale, branched vascular network with an interpenetrated airway network, which can be fabricated by using the bioprinter in its basic configuration. This model is prepared in CAD software and then exported into an “.stl” format. The exported file is uploaded to a software and the print parameters are entered. Once the pre-polymerization solution is prepared, it is transferred onto the vat housed on the build platform. Then, the Z-axis stage is lowered to obtain the desired layer thickness. A series of automated projections of the 3D model and Z-stage translations ultimately results in the final 3D printed hydrogel. The 3D bioprinted hydrogel can then be removed from the Z-platform. The vascular channel of the 3D printed model can be endothelialized by perfusion of endothelial cells while the airway channel can be epithelialized by perfusion of epithelial cells. The vascular network serves as the blood supply, delivering oxygen and nutrients to the cells in the bulk of the hydrogel while the airway network provides a means to supply oxygen to the vascular network.

Returning to FIG. 1, in various embodiments, a crosslinker or crosslinking agent 130 may be introduced into the open channel 120 of the target material 110 and allowed to diffuse into the target material 110 beyond the open channel 120, after which excess cross-linking agent is flushed out leaving the diffused crosslinking agent 140 that has diffused into the target material 110. An example optimized but non-limiting formulation for the crosslinking agent 130 consists of bovine thrombin dissolved in phosphate buffered saline (e.g., pH range of between about 7.4 to about 8) to a concentration of between about 5 Units/mL to about 20 Units/mL, at a temperature of between about 20° C. to about 40° C. or between about 35° C. to about 37° C.

In some embodiments, to confine polymerization to the channel 120 interface, the crosslinking agent 130 may be introduced into the channel 130 of the target material 110 prior to the carrier composition 150 (e.g., and allowed to diffuse into the target material 110 beyond the open channel 120) resulting in the diffused cross-linking channel 140. After the introduction of the cross-linking agent 130 (e.g., and its diffusion into the target material 110 resulting in the diffused cross-linking agent 140 and the flushing out of the excess cross-linking agent), a carrier composition 150 may be flown into the open channel 120 that is surrounded by the diffused cross-linking agent 140. In various embodiments, the carrier composition 150 may be or include a pre-hydrogel solution including cells (e.g., ECs), beads, particles, and/or etc., and a polymerizable material (e.g., and in some instances, without a cross-linking agent).

In some embodiments, a cross-linking agent 130 may be introduced into the open channel 120 of the target material 110 as part of the carrier composition 150 and a polymerizable material may be introduced into the open channel separately. That is, a polymerizable material may be introduced into the target material 110 (e.g., or open channel 120 thereof) and then, a carrier composition including a cross-linking agent 130 and cells, beads, and/or particles may be introduced into the open channel 120 of the target material 110, or vice versa.

Non-limiting example combinations of polymerizable materials and cross-linking agents 130 that crosslink said polymerizable materials include fibrinogen enzymatically crosslinked by thrombin or ancrod, a snake venom-derived enzyme that cleaves only FpA and not FpB, contrary to thrombin which cleaves both FpA and FpB. Said polymerizable material and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable material, and (ii) cells, beads, particles, etc., may be incubated at various concentrations, pH, and/or temperature to cause crosslinking of the polymerizable materials. For example, thrombin or ancrod may be used at a concentration of between about 0.1 units /mL to about 25 units/mL, between about 1 units/mL to about 25 units/mL or between about 5 units/mL to about 20 units/mL. Fibrinogen can be used at a concentration of between about 5 mg/mL to about 25 mg/mL, between about 10 mg/mL to about 20 mg/mL or between about 10 mg/mL to about 15 mg/mL. The pH may be set at the about 6-8 range, particularly at about 7-7.4 range. Aqueous solutions can include but are not limited to cell culture media or phosphate buffered saline (PBS), (4-(2-hydroxyethyl)-1-piperazineethanesulfonic acid (HEPES), piperazine-N,N′-bis(2-ethanesulfonic acid) (PIPES), 1,4-bis(2-hydroxyethyl)piperazine (BHEP), or Tris (2-amino-2-hydroxymethylpropane-1,3-diol) (Tris), and sodium bicarbonate, with PBS as a particular buffer choice. Any of these buffers may be adjusted to about pH 7.4, and the incubation temperatures, i.e., the crosslinking temperatures, can be set in the range from about 20° C. to about 40° C., from about 30° C. to about 39° C., from about 35° C. to about 37° C., including values and subranges therebetween.

Another example combination of polymerizable materials and cross-linking agents 130 that crosslink said polymerizable materials includes gelatin or gelatin methacrylate enzymatically crosslinked by transglutaminase. Said Polymerizable materials and a carrier composition including (i) transglutaminase, and (ii) cells, beads, particles, etc., may be incubated at various concentrations, pH, and/or temperature to cause crosslinking of the polymerizable materials. As another example, transglutaminase and a carrier composition including (i) said polymerizable materials, and (ii) cells, beads, particles, etc., may be incubated at various concentrations, pH, and/or temperature to cause crosslinking of the polymerizable materials. For example, gelatin or gelatin methacrylate may be used at concentrations of between about 1 wt % to about 20 wt %, about 1 wt % to about 10 wt %, about 1 wt % to about 5 wt %, including values and subranges therebetween. Transglutaminase may be used at concentration of between about 1 units/g to about 50 units/g, between about 5 units/g to about 40 units/g, between about 10 units/g to about 30 units/g gelatin, between about 15 units/g to about 25 units/g, including values and subranges therebetween. Aqueous solution includes cell culture media or buffers listed above. The incubation temperatures, i.e., crosslinking temperatures, can be set at between about 25° C. to about 70° C., about 30° C. to about 50° C., about 25° C. to about 35° C., about 35° C. to about 37° C., including values and subranges therebetween.

Another example combination of polymerizable materials and cross-linking agents 130 that crosslink said polymerizable materials includes silk fibroin crosslinked enzymatically by peroxidases, including but not limited to a solution of hydrogen peroxide and horseradish peroxidase. Said Polymerizable materials and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable materials, and (ii) cells, beads, particles, etc., may be incubated at various concentrations, pH, and/or temperature to cause crosslinking of the polymerizable materials. For example, hydrogen peroxide can be used between about 0.5 mM to about 20 mM, between about 1 mM to about 10 mM, between about 1 mM to about 5 mM, etc., and horseradish peroxidase can be used at between about 5 to about 50 Units/mL, between about 10 Units/mL to about 25 Units/mL, etc., in phosphate buffered saline at pH of 7.4. The concentration of silk fibroin can be between about 2 wt % to about 20 wt %, about 5 wt % to about 10 wt %, etc. The incubation temperatures, i.e., crosslinking temperatures, can be set between about 20° C. to about 40° C., about 25° C. to about 70° C., about 30° C. to about 50° C., about 25° C. to about 35° C., about 35° C. to about 37° C., including values and subranges therebetween.

Another example combination of polymerizable materials and cross-linking agents 130 that crosslink said polymerizable materials includes silk fibroin crosslinked with light actvated photoinitiators, including but not limited to ruthenium. In various embodiments, a waveguide may be used to limit exposure of the cross-linking agent to light activation. Silk fibroin and a carrier composition including (i) photoinitiators, and (ii) cells, beads, particles, etc., may be incubated at various concentrations, pH, and/or temperature to cause crosslinking of silk fibroin. As another example, photoinitiators and a carrier composition including (i) silk fibroin, and (ii) cells, beads, particles, etc., may be incubated at various concentrations, pH, and/or temperature to cause crosslinking of silk fibroin. For example, ruthenium at between about 0.1 mM to about 10 mM or between about 1 mM to about 5 mM, etc., or riboflavin at between about 0.05 mM to about 20 mM or between about 0.5 mM to about 2.5 mM, etc., can be used in combination with sodium persulfate at between about 1 mM to about 100 mM or between about 10 mM to about 25 mM, etc. Aqueous solutions for the incubation include buffers PBS or HEPES at about pH 7.5. The concentration of silk fibroin can be between about 2 wt % to about 20 wt %, about 5 wt % to about 10 wt %, etc. The incubation temperatures, i.e., crosslinking temperatures, can be set at between about 20° C. to about 40° C., between about 25° C. to about 70° C., between about 30° C. to about 50° C., between about 25° C. to about 35° C., between about 35° C. to about 37° C., including values and subranges therebetween.

Another example combination of polymerizable materials and cross-linking agents 130 that crosslink said polymerizable materials includes alginate ionicially crosslinked by Ca²⁺ ions or Ba²⁺ ions. Said polymerizable material and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable material, and (ii) cells, beads, particles, etc., may be incubated at various concentrations, pH, and/or temperature to cause crosslinking of the polymerizable materials. For example, the incubation conditions may include alginate concentrations of between about 0.5 wt % to about 2 wt %, between about 1 wt % to about 1.5 wt %, etc., in aqueous solutions or buffers listed above. The concentrations of calcium chloride or barium chloride can be between about 50 mM to about 500 mM, about 100 mM to about 250 mM, about 100 mM to 150 mM, etc., in aqueous solutions or buffers listed above. The incubation temperatures, i.e., crosslinking temperatures, can be set at between about 20° C. to about 40° C., between about 25° C. to about 70° C., between about 30° C. to about 50° C., between about 25° C. to about 35° C., between about 35° C. to about 37° C., including values and subranges therebetween.

Another example combination of polymerizable materials and cross-linking agents 130 that crosslink said polymerizable materials includes acrylate, methacrylate, acrylamide or methacrylamide functionalized polymers crosslinked by ammonium persulfate (APS) and tetramethylethylenediamine (TEMED). Said polymerizable materials and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable materials, and (ii) cells, beads, particles, etc., may be incubated at various concentrations, pH, and/or temperature to cause crosslinking of the polymerizable materials. For example, polymers include but are not limited to gelatin methacryloyl (GelMA), polyethylene glycol diacrylate (PEGDA), methacrylated hyaluronic acid, or collagen methacrylate at concentrations of between about 1 wt % to about 20 wt %. APS concentrations can be at between about 0.1 wt % to about 1 wt %, about 0.2 wt % to about 0.5 wt %, etc. TEMED concentrations ca be at between about 0.05 wt % to about 1 wt %, between about 0.1 wt % to about 0.5 wt %, etc. The incubation temperatures, i.e., crosslinking temperatures, can be between about 20° C. to about 40° C., between about 25° C. to about 70° C., between about 30° C. to about 50° C., between about 25° C. to about 35° C., between about 35° C. to about 37° C., including values and subranges therebetween.

Another example combination of polymerizable materials and cross-linking agents 130 that crosslink said polymerizable materials includes click-chemistry pairs includes PEG-dithiol, PEG8-norbornene, thiolated gelatin, thiolated chitosan, thiolated silk, thiolated decellularized ECM, or combinations thereof. Said polymerizable materials and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable materials, and (ii) cells, beads, particles, etc., may be incubated at various concentrations, pH, and/or temperature to cause crosslinking of the polymerizable materials. For example, two-component click chemistry reactions involving an ene and thiol component can be used, where ene components include but are not limited to 4-arm or 8-arm PEG-norbornene or norbornene-functionalized gelatin or peptide sequences at concentrations of about 1 wt % to about 20 wt %, about 5 wt % to about 10 wt %, etc., and thiol components include but are not limited to PEG-dithiol, 4-arm PEG-thiol, thiolated gelatin, dithiothreitol, or di-cysteine terminated peptide sequences at concentrations of between about 1 wt % to about 20 wt %, about 5 wt % to about 10 wt %, etc. For the incubation, aqueous solutions including but are not limited to buffers such as PBS or HEPES with pH of between about 7 to about 13, between about 7.4 to about 8, etc., can be used. Another example click-chemistry pair cross-linking includes supramolecular guest-host-type crosslinking, including but not limited to 4-arm or 8-arm PEG-adamantane and 4-arm or 8-arm PEG-cyclodextrin. In various embodiments, the components may be dissolved in aqueous solution or buffer as listed above, concentration of between about 1 wt % to about 20 wt %, about 5 wt % to about 10 wt %, etc. The incubation temperatures, i.e., crosslinking temperatures, can be between about 20° C. to about 40° C., between about 25° C. to about 70° C., between about 30° C. to about 50° C., between about 25° C. to about 35° C., between about 35° C. to about 37° C., including values and subranges therebetween.

Another example combination of polymerizable materials and cross-linking agents 130 that crosslink said polymerizable materials includes acrylate, methacrylate, acrylamide, or methacrylamide functionalized polymers crosslinked by cysteine-terminated peptide sequences. Said polymerizable materials and a carrier composition including (i) said cross-linking agents, and (ii) cells, beads, particles, etc., may be incubated at various concentrations, pH, and/or temperature to cause crosslinking of the polymerizable materials. As another example, said cross-linking agents and a carrier composition including (i) said polymerizable materials, and (ii) cells, beads, particles, etc., may be incubated at various concentrations, pH, and/or temperature to cause crosslinking of the polymerizable materials. For example, polymers include but are not limited to polyethylene glycol diacrylate, polyethylene glycol methacrylate, polyethylene glycol acrylamide, gelatin methacryloyl at concentrations of between about 1 wt % to about 20 wt %, about 5 wt % to about wt %, etc. Cysteine-terminated peptide sequences include CGPQGIWGQGCR, CGPQGIAGQGCR, or CGPQGPAGQGCR at concentrations of between about 1 wt % to about 10 wt %. The incubation may occur at pH of between about 7 to about 13, about 7.4 to about 8, etc. The incubation temperatures, i.e., crosslinking temperatures, can be between about 20° C. to about 40° C., between about 25° C. to about 70° C., between about 30° C. to about 50° C., between about 25° C. to about 35° C., between about 35° C. to about 37° C., including values and subranges therebetween.

Examples of light-polymerizable materials crosslinked by light include acrylate, methacrylate, or acrylamide functionalized polymers. Such polymerizable materials introduced in the open channel of the target materials (e.g., as part of a pre-hydrogel solution or separately) may be incubated at various concentrations, pH, and/or temperature to cause crosslinking of the light-polymerizable materials. For example, polymers that include but are not limited to polyethylene glycol diacrylate, gelatin methacryloyl, collagen methacrylate, silk methacrylate, methacrylated hyaluronic acid, etc., can have concentrations of between about 1 wt % to about 20 wt %, 5 wt % to about 10 wt %, etc. Photoinitiators can include but are not limited to Irgacure 2959, eosin Y, or lithium acylphosphinate at concentrations of between about 0.01 wt % to about 1 wt %, about 0.05 wt % to about 0.1 wt %, etc. Aqueous solution used for incubation include but are not limited to cell culture media or buffer such as PBS or HEPES with pH of about 7.4. The incubation temperatures, i.e., crosslinking temperatures, can be between about 20° C. to about 40° C., between about 25° C. to about 70° C., between about 30° C. to about 50° C., between about 25° C. to about 35° C., between about 35° C. to about 37° C., including values and subranges therebetween. Some photoinitiators may require additional initiator, such as triethanolamine (TEA), at concentrations of between about 0.01 wt % to about 2 wt % or between about 0.05 wt % to about 0.25 wt %, and another crosslinker such as 1-vinyl-2-pyrrolidinone (NVP), at concentrations of between about 10 nM to about 100 nM , about 25 nM to about 50 nM, etc.

In the embodiments where the polymerizable materials are cross-linked due to light, the incident light can be guided along the vascular network using the pre-hydrogel as a liquid-phase waveguide. In such cases, pre-hydrogel solutions with an index of refraction greater than the primary hydrogel can be used such that light can totally internally reflect through the vascular channels and penetrate deep into the gel while limiting phototoxicity.

In various embodiments, the polymerizable materials may be pH-polymerizable, temperature-polymerizable, etc., and in such cases, a cross-linking agent 130 may not be used to polymerize the polymerizable materials. For example, with reference to FIG. 1, the carrier composition 150 flown into the open channel 120 may be or include a pre-hydrogel solution including cells, beads, particles, and/or etc., and a polymerizable material, without a cross-linking agent. In some instances, the polymerizable material and the pre-hydrogel solution including cells, beads, particles, and/or etc., may flow into the open channel 120 separately, without a cross-linking agent. In such cases, the pH-, temperature-, etc., polymerizable materials in the open channel 120 may be crosslinked due to exposure to pH (e.g., pH gradient), and/or temperature (e.g., temperature gradient), etc., respectively.

Examples of pH-polymerizable materials include collagen self-assembly induced by pH gradient; peptide-based hydrogels and peptide amphiphiles. Such polymerizable materials introduced in the open channel of the target materials (e.g., as part of a pre-hydrogel solution or separately) may be incubated at various concentrations, pH, and/or temperatures to cause crosslinking of the pH-polymerizable materials. For example, collagen can have concentrations of between about 1 mg/mL to about 10 mg/mL, about 1 mg/mL to about 2 mg/mL, etc., in aqueous buffers as listed above, and may be maintained at acidic or neutral pH (e.g., between about 3 to about 7 or between about 5 to about 6). In various embodiments, pH gradient can be established by using an alkaline solution as the crosslinking solution, for example, any of the above-listed buffer solutions with added sodium hydroxide can be used to yield a final pH in the range of between about 7 to about 10, about 8 to about 9, etc. The incubation temperatures, i.e., crosslinking temperatures, can be between about 20° C. to about 40° C., between about 25° C. to about 70° C., between about 30° C. to about 50° C., between about 25° C. to about 35° C., between about 35° C. to about 37° C., including values and subranges therebetween.

Examples of temperature-polymerizable materials include agarose where the material polymerizes as its temperature is lowered. Such polymerizable materials introduced in the open channel of the target materials (e.g., as part of a pre-hydrogel solution or separately) may be incubated at various concentrations, pH, and/or temperatures to cause crosslinking of the temperature-polymerizable materials. For example, agarose can have concentrations of between about 1 wt % to about 4 wt %, about 1 wt % to about 2 wt %, etc., in aqueous buffer as listed above. And temperature gradient that causes the polymerization can be achieved for agarose by pre-cooling the target material in the range of between about 4° C. to about 25° C., about 4° C. to about 10° C., etc., then injecting agarose solution at a temperature of between about 25° C. to about-50° C., about 35° C. to about 37° C., etc.

Another example of temperature-polymerizable materials include Matrigel® where the material polymerizes as its temperature is raised. Such polymerizable materials introduced in the open channel of the target materials (e.g., as part of a pre-hydrogel solution or separately) may be incubated at various concentrations, pH, and/or temperatures to cause crosslinking of the temperature-polymerizable materials. For example, Matrigel® can have concentrations of between about 1 wt % to about 20 wt %, about 5 wt % to about 7 wt %, etc., in aqueous buffer, maintained on ice. And temperature gradient that causes the polymerization can be achieved for Matrigel® by pre-warming the target material in the range of between about 25° C. to about 50° C., about 35° C. to about 37° C., etc., then injecting Matrigel® solution at a temperature of between about 0° C. to about 25° C., about 0° C. to about 4° C., etc.

In various embodiments, to prevent cells from settling during the crosslinking process, a thickener is added to increase the viscosity of the cell containing solution. Thickeners include but are not limited to xanthan gum, guar gum, or gellan gum with concentrations of between about 0.01 wt % to about 2 wt %, about 0.05 wt % to about 0.1 wt %, etc. Other thickeners can include but are not limited to gelatin, polyvinyl alcohol, or polyethylene glycol (>10,000 Da) at concentrations of between about 10 wt % to about 50 wt %, about 10 wt % to about 20 wt %, etc.

As discussed above, the carrier composition 150 includes cells, beads, particles, etc. In various embodiments, the carrier composition 150 may also include other biological and/or pharmaceutical materials such as but not limited to sensors, labels, active substances (e.g., protein, nuclei, etc.), etc.

In various embodiments, the cells in the carrier composition 150 can be of almost any nature and have chemical makeup, and have size in ranging from about 100 nm to about 100 μm. Examples include but are not limited to bacterial cells, fungal cells, plant cells, animal cells, and/or the like. For example, the animal cells may be mammalian cells, such as human or non-human cells (e.g., dog, cat, rabbit, horse cow, mouse, rat, non-human primate, etc.). Particular types of cells include muscle cells (e.g., smooth muscle cells), endothelial cells (e.g., vascular endothelial cells), epithelial cells, mesodermal cells, immune cells, liver cells, pancreatic cells, lung cells, neuronal cells, skin cells, retinal cells, corneal cells, fibroblasts, stem cells, or lymphatic endothelial cells.

In various embodiments, the cells may be tumor cells. For example, the techniques disclosed herein can be used to create or manufacture artificial tumors, including those with their own supporting vasculature, for studies on the application of drugs for treatment of cancer. In various embodiments, cells can be obtained directly from a donor, from cell culture of cells from a donor, or from established cell culture lines. In particular embodiments, cells are obtained directly from a donor, washed and used without culture. Cultured cells may be cultured using techniques known to those skilled in the art of tissue culture.

In various embodiments, cell viability can be assessed using standard techniques, such as histology and fluorescent microscopy. The function of the implanted cells can be determined using a combination of these techniques and functional assays. For example, in the case of hepatocytes, in vivo liver function studies can be performed by placing a cannula into the recipient's common bile duct. Bile can then be collected in increments. Bile pigments can be analyzed by high pressure liquid chromatography looking for underivatized tetrapyrroles or by thin layer chromatography after being converted to azodipyrroles by reaction with diazotized azodipyrroles ethylanthranilate either with or without treatment with P-glucuronidase. Diconjugated and monoconjugated bilirubin can also be determined by thin layer chromatography after alkalinemethanolysis of conjugated bile pigments. In general, as the number of functioning transplanted hepatocytes increases, the levels of conjugated bilirubin will increase. Simple liver function tests can also be done on blood samples, such as albumin production. Analogous organ function studies can be conducted using techniques known to those skilled in the art, as required to determine the extent of cell function after implantation. For example, pancreatic islet cells and other insulin-producing cells can be implanted to achieve glucose regulation by appropriate secretion of insulin. Other endocrine tissues and cells can also be implanted.

In various embodiments, the amount and density of cells included in the carrier composition 150 for coating a target material 110 may vary depending on the choice of cell, surface and intended use. In some embodiments, the cells are at a concentration of between about 0.1×10⁶ cells/mL to about 100×10⁶ cells/mL, about 10×10⁶ cells/mL to about 50×10⁶ cells/ml, etc., in the carrier. In some embodiments, the injected material can include a combination of cells (such as smooth muscle cells and endothelial cells, at ratios of between about 1:1 to about 1:10, about 1:1 to 1:5, etc.). In other embodiments, the cells are present as cell aggregates with either a single cell type or a combination of cell types. Cells can be harvested from culture on cell culture treated substrates or used from frozen bullets in which the cells are thawed, rinsed, then resuspended with the desired cell carrier material.

In various embodiments, the beads and particles in the carrier composition 150 include non-cellular materials such as but not limited to liposomes, nanoparticles, magnetic beads, polystyrene beads, PEG or gelatin microspheres, any polymeric microspheres, and/or the like. These materials may be derivatized with agents including labels, therapeutics, biological agents, chemical agents, sensors, etc.

As mentioned above, the carrier composition 150 may also include biological and/or pharmaceutical materials such as but not limited to sensors, labels, active substances (e.g., proteins, nuclei, etc.), etc. For example, the biological and/or pharmaceutical materials can be any compound, composition, conjugate, or construct that can be used to diagnose or treat a disease, disorder, condition, symptom, etc. Examples of such materials or agents include cells, tissues, cell products, tissue products, proteins, antibodies, vaccines, vaccine components, antigens, epitopes, drugs, salts, nutrients, buffers, acids, bases, and/or the like. Additional examples of biological materials include any biological substance such as but not limited to biological micromolecules (e.g., nucleotides, amino acids, cofactors, hormones, etc.) or biological macromolecules (e.g., nucleic acids, polypeptides, proteins, polysaccharides, etc.). Examples of proteins include enzymes, receptors, secretory proteins, structural proteins, signaling proteins, hormones, ligands, etc. Any class, type, form, or particular biological material can be used together with any other classes, types, forms, or particular biological materials.

In various embodiments, the cells, beads, particles, etc., may contain biological sensor molecules or systems including but not limited to oxygen sensors, glucose sensors, pH sensors, heat sensors, etc. In some instances, the cells may be the sensor themselves, such as a cell that is genetically manipulated to produce a fluorescent protein in the presence of an agent, for example, oxygen or glucose. In various embodiments, the cells, beads, particles, etc., may also contain a label that permits detection/quantitation of the material. Labels include radiolabels, dyes, fluorescent molecules, chemiluminescent molecules, a ligand tag, etc., with affinity for a ligand binding molecule, such as biotin-avidin, hybridizing nucleic acid probe pairs, antibody-antigen pairs, etc.

In various embodiments, the interfacial layer itself, or the cells, beads, particles, biological and/or pharmaceutical materials, etc., embedded therein, may contain other active substances. For example, as discussed above, a therapeutically effective substance, such as a protein or nucleic acid may be included. In some embodiments, the cells produce a metabolic product. In some embodiments, the cells metabolize toxic substances. In some embodiments, the cells form structural tissues, such as skin, bone, cartilage, blood vessels, or muscle. In some embodiments, the cells are natural, such as islet cells that naturally secrete insulin, or hepatocytes that naturally detoxify. In some embodiments, the cell produces or secretes a factor that promotes or inhibits immobilization. In some embodiments, the cells are genetically engineered to express a heterologous protein or nucleic acid and/or overexpress an endogenous protein or nucleic acid. In some embodiments, the cells are genetically engineered to produce a new or different product, which can be an expression product of the engineered gene(s) or another product, such as a metabolite, produced because of the engineered gene(s).

Examples of therapeutic agents that can be included in the interfacial layer, or engineered into cells included in the interfacial layer, comprise thyroid stimulating hormone; beneficial lipoproteins such as Apol; prostacyclin and other vasoactive substances, anti-oxidants and free radical scavengers; soluble cytokine receptors, for example soluble transforming growth factor (TGF) receptor, or cytokine receptor antagonists, for example IL1ra; soluble adhesion molecules, for example ICAM-1; soluble receptors for viruses, e.g., CD4, CXCR4, CCR5 for HIV; cytokines; elastase inhibitors; bone morphogenetic proteins (BMP) and BMP receptors 1 and 2; endoglin; serotonin receptors; tissue inhibiting metaloproteinases; potassium channels or potassium channel modulators; anti-inflammatory factors; angiogenic factors including vascular endothelial growth factor (VEGF), transforming growth factor (TGF), hepatic growth factor, and hypoxia inducible factor (HIF); polypeptides with neurotrophic and/or anti-angiogenic activity including ciliary neurotrophic factor (CNTF), glial-derived neurotrophic factor (GDNF), nerve growth factor (NGF), brain-derived neurotrophic factor (BDNF), neurotrophin-3, nurturin, fibroblast growth factors (FGFs), endostatin, ATF, fragments of thrombospondin, variants thereof and/or the like. More particular polypeptides are FGFs, such as acidic FGF (aFGF), basic FGF (bFGF), FGF-1 and FGF-2 and endostatin.

In various embodiments, the active agent can be a protein or peptide. Examples of protein active agents include, but are not limited to, cytokines and their receptors, as well as chimeric proteins including cytokines or their receptors, including, for example tumor necrosis factor alpha and beta, their receptors and their derivatives; renin; lipoproteins; colchicine; prolactin; corticotrophin; vasopressin; somatostatin; lypres sin; pancreozymin; leuprolide; alpha-1-antitrypsin; clotting factors such as factor VIIIC, factor IX, tissue factor, and von Willebrand's factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1 -alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-a and TGF-β, including TGF-βI, TGF-2, TGF-3, TGF-4, or TGF-5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon alpha 2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; transport proteins; homing receptors; addres sins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of protein active agents also include CCL1, CCL2 (MCP-1), CCL3 (MIP-Ia), CCL4 (MIP-I(3), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD4OLG), TNFSF6, TNFSF7, TNFSF8, TNFSF9, TNFSF10, TNFSF11, TNFSF13B, EDA, IL2, IL15, IL4, IL13, IL7, IL9, IL21, IL3, ILS, IL6, IL1 1, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, ILIA (IL1F1), IL1B (IL1F2), ILIRa (IL1F3), IL1F5 (IL36RN), IL1F6 (IL36A), IL1F7 (IL37), IL1F8 (IL36B), IL1F9 (IL36G), ILIFIO (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp1O, Bmp15, Bmp2, Bmp3, Bmp4, Bmp5, Bmp6, Bmp7, Bmp8a, Bmp8b, Clqtnf4, Cc121a, Cc127a, Cd70, Cer1, Ck1f, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf1O, Gdfl 1, Gdfl5, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifnal 1, Ifnal2, Ima9, Ifnab, Ifne, 1117a, 1123a, 1125, 1131, Iltifb, Inhba, Lefty1, Lefty 2, Mstn, Nampt, Ndp, Nodal, Pf4, Pgly 1, Prl7d1, Scg2, Scgb3a1, Slurp1, Spp1, Thpo, Tnfsf1O, Tnfsfl 1, Tnfsfl2, Tnfsfl3, Tnfsfl3b, Tnfsfl4, Tnfsfl5, Tnfsfl8, Tnfsf4, Tnfsf8, Tnfsf9, Tslp, Vegfa, Wnt1, Wnt2, Wnt5a, Wnt7a, Xcl1, Epinephrine, Melatonin, Triiodothyronine, Thyroxine, Prostaglandins, Leukotrienes, Prostacyclin, Thromboxane, Islet Amyloid Polypeptide, Miillerian inhibiting factor or hormone, Adiponectin, Corticotropin, Angiotensin, vasopressin, arginine vasopressin, atriopeptin, Brain natriuretic peptide, Calcitonin, Cholecystokinin, Cortistatin, Enkephalin, Endothelin, Erythropoietin, Follicle-stimulating hormone, Galanin, Gastric inhibitory polypeptide, Gastrin, Ghrelin, Glucagon, Glucagon-like peptide-1, Gonadotropin-releasing hormone, Growth hormone-releasing hormone, Hepcidin, Human chorionic gonadotropin, Human placental lactogen, Growth hormone, Inhibin, Insulin, Somatomedin, Leptin, Lipotropin, Luteinizing hormone, Melanocyte stimulating hormone, Motilin, Orexin, Oxytocin, Pancreatic polypeptide, Parathyroid hormone, Pituitary adenylate cyclase-activating peptide, Prolactin, Prolactin releasing hormone, Relaxin, Renin, Secretin, Somatostatin, Thrombopoietin, Thyrotropin, Thyrotropin-releasing hormone, Vasoactive intestinal peptide, Androgen, Androgen, acid maltase (alpha-glucosidase), glycogen phosphorylase, glycogen debrancher enzyme, Phosphofructokinase, Phosphogly cerate kinase, Phosphogly cerate mutase, Lactate dehydrogenase, Carnitine palymityl transferase, Carnitine, Myoadenylate deaminase, and/or the like.

Additional examples of biological and/or pharmaceutical materials include hormones that are included into the carrier composition 150 or produced by the cells in the carrier composition 150. Examples of such hormones include endocrine hormones such as but not limited to anti-diuretic hormone (ADH), which is produced by the posterior pituitary, targets the kidneys, and affects water balance and blood pressure; oxytocin, which is produced by the posterior pituitary, targets the uterus, breasts, and stimulates uterine contractions and milk secretion; growth hormone (GH), which is produced by the anterior pituitary, targets the body cells, bones, muscles, and affects growth and development; Prolactin, which is produced by the anterior pituitary, targets the breasts, and maintains milk secretions; growth hormone-releasing hormone (GHRH), which is a releasing hormone of GH and is produced in the arcuate nucleus of the hypothalamus; thyroid stimulating hormone (TSH), which is produced by the anterior pituitary, targets the thyroid, and regulates thyroid hormones; thyrotropin-release hormone (TRH), which is produced by the hypothalamus and stimulates the release of TSH and prolactin from the anterior pituitary; adrenocorticotropic hormone (ACTH), which is produced by the anterior pituitary, targets the adrenal cortex, and regulates adrenal cortex hormones; follicle-stimulating hormone (FSH), which is produced by the anterior pituitary, targets the ovaries/testes, and stimulates egg and sperm production; luteinizing hormone (LH), which is produced by the anterior pituitary, targets the ovaries/testes, and stimulates ovulation and sex hormone release; luteinizing hormone-releasing hormone (LHRH), also known as gonadotropin-releasing hormone (GnRH), which is synthesized and released from GnRH neurons within the hypothalamus and is a trophic peptide hormone responsible for the release of FSH and LH; Thyroxine, which is produced by the thyroid, targets the body cells, and regulates metabolism; Calcitonin, which is produced by the thyroid, targets the adrenal cortex, and lowers blood calcium; parathyroid hormone, which is produced by the parathyroid, targets the bone matrix, and raises blood calcium; aldosterone, which is produced by the adrenal cortex, targets the kidney, and regulates water balance; cortisol, which is produced by the adrenal cortex, targets the body cells, and weakens immune system and stress responses; epinephrine, which is produced by the adrenal medulla, targets the heart, lungs, liver, and body cells, and affects primary “fight or flight” responses; glucagon, which is produced by the pancreas, targets the liver body, and raises blood glucose level; insulin, which is produced by the pancreas, targets body cells, and lowers blood glucose level; estrogen, which is produced by the ovaries, targets the reproductive system, and affects puberty, menstrual, and development of gonads; progesterone, which is produced by the ovaries, targets the reproductive system, and affects puberty, menstrual cycle, and development of gonads; and testosterone, which is produced by the adrenal gland, testes, targets the reproductive system, and affects puberty, development of gonads, and sperm.

In various embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II); and/or the like.

Returning to FIG. 1, the cross-linking agent 140 that has diffused into the target material 110 may react with the carrier material 150 along the walls 170 of the open channel 120 leading to a local polymerization reaction which entraps or immobilizes the cells (e.g., ECs), beads, particles, etc., 160 in the carrier material 150. Because the cross-linking agent 140 comes into contact with the carrier material 150 only at the channel interface 170, the reaction is confined to this region. To prevent complete polymerization of the carrier material 150 (which would occlude the channel 120), the crosslinking reaction can be timed and excess carrier material may be flushed out before it can be polymerized. As a non-limiting example illustration, the carrier material 150 can be exposed to the target material 110 by flowing the carrier material 150 via the open channel 120 at a rate of between about 0.1 mL/min to about 10 mL/min, about 1 mL/min to about 2.5 mL/min, about 0.5 mL/min to about 5 mL/min, about 2.5 mL/min to about 7.5 mL/min, about 1 mL/min to about 5 mL/min, including values and subranges therebetween. Further, the injection and incubation in the target material 110 of the carrier material (e.g., including the polymerizable material and the cells, beads, particles, etc.) and the cross-linking agent, or the injection and incubation in the target material 110 of the carrier material (e.g., including the cross-linking agent and the cells, beads, particles, etc.) and the polymerizable material can be done at incubation temperatures between about 20° C. to about 40° C., between about 25° C. to about 70° C., between about 30° C. to about 50° C., between about 25° C. to about 35° C., between about 35° C. to about 37° C., including values and subranges therebetween. In various embodiments, the incubating and the washing out of excess composition occur in less than 1 hour, less than 45 mins, less than 30 mins, less than 25, mins, less than 20 mins, less than 15 mins, less than 12 mins, less than 11 mins, less than 10 mins, less than 9 mins, less than 8 mins, less than 7 mins, less than 6 mins, less than 5 mins, less than 4 mins, less than 3 mins, or less than 2 mins, but occur for at least 1 min.

Example Applications of Techniques Disclosed Herein

The techniques disclosed herein can be employed in a number of particular applications, including to manufacture cell coated surfaces that may be used to create artificial membranes, tissues and organs that may be utilized in laboratory research both in vitro and in vivo in experimental animals and for clinical studies in patients, including humans, for both transplant and reconstructive purposes. Examples include creating artificial tissues and vascular complexes, including arterial, venous and lymphatic—for reconstruction of damaged tissues, as well as generating entirely engineered organs. Other examples of applications include the manufacturing of devices, including those suitable for implantation into subjects, that can monitor the subjects' health and physiologic condition, as well as deliver therapeutic agents, non-limiting examples including devices configured to monitor glucose and produce/deliver insulin. Additional examples of applications include production of so-called “vessel-on-a-chip” devices that can be used for rapid and scalable drug screening or for mimicking vascular networks and/or tissues to determine the efficacy of therapies or the potentially toxic effects of drugs or environmental contaminants.

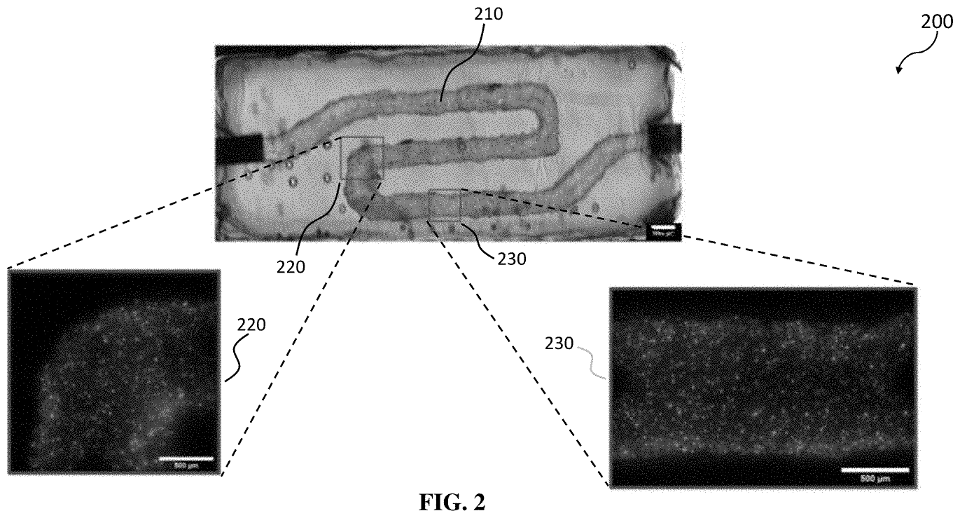

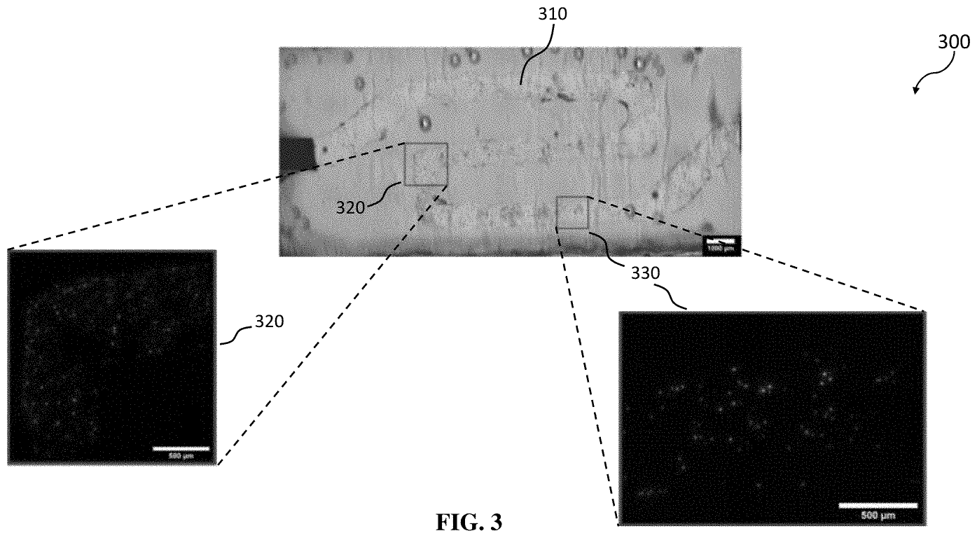

FIGS. 2 and 3 show example respective illustrations 200 and 300 of the application of techniques disclosed herein where rapid endothelialization of patterned vascular channels is effected via interfacial gel polymerization in the presence and absence, respectively, of a cross-linking agent, according to various embodiments. In various embodiments, A549 epithelial-like cells are used as a proxy for ECs, i.e., A549 were seeded in fibrinogen pre-hydrogel solution (10 mg/mL), and about 5 mins of crosslinking of the cell-laden fibrinogen with thrombin crosslinker was found to be sufficient to entrap or immobilize a uniform layer of cells around the circumference of the channels 210, 310 in a planar serpentine architecture. Addition of xanthan to the cell-laden fibrinogen increases the density of the suspension to prevent cell settling and inhomogeneous distribution of entrapped cells. Broadly, the thickness of the interfacial layer may be between about 10 μm to about 1000 μm. In some embodiments where the polymerized layer contains endothelial or epithelial cells, the optimal thickness of the polymerized layer may be approximately equal to one cell length, approximately about 30 μm to about 60 μm. The thickness of the interfacial layer may be modulated by the concentrations of the crosslinker and carrier material, the duration of the crosslinker incubation period and crosslinking reaction, and the temperature of the system.

Comparison of FIG. 2 and FIG. 3 shows that the presence of cells along the lumenal surfaces of the channels 210 and 310 can depend on the presence a cross-linking agent, implying that the vast majority of cells lining the channels 210 are entrapped in the polymerized layer and not adhered on the surface of the underlying channel 210. For example, FIG. 2, corresponding to the presence of a cross-linking agent in the channel 210 (i.e., pre-incubation presence of thrombin in the channel 210), shows the presence of cells within, and in particular, along the lumenal surface of the channel 210 (e.g., as shown in the portions 220 and 230 that are magnified), while FIG. 3, corresponding to the absence of a cross-linking agent in the channel 310, shows sparse cells within, and in particular, along the lumenal surface of the channel 310 (e.g., as shown in the portions 320 and 330 that are magnified).

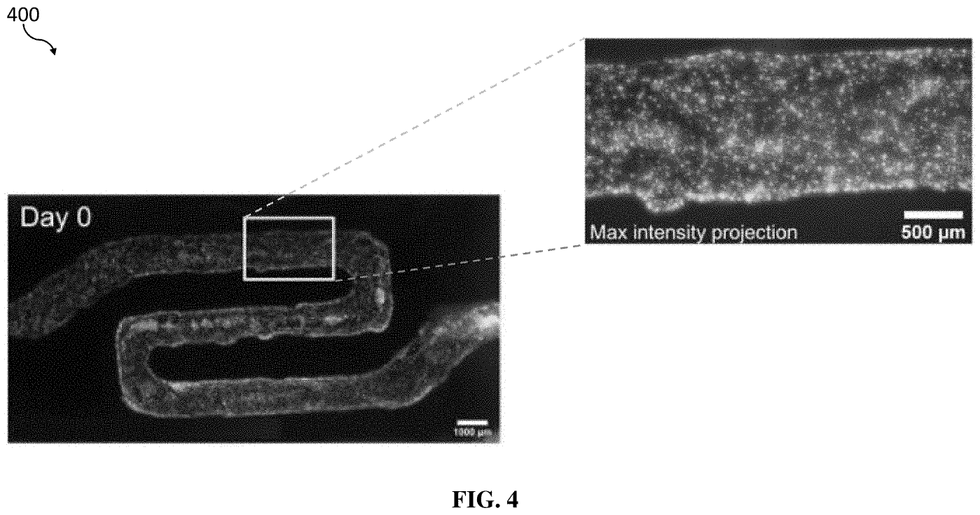

FIGS. 4-7B show example luminal cell morphogenesis over several days following seeding through interfacial polymerization, according to various embodiments. Establishing a layer of cells along the surface of a channel is necessary, but not sufficient, for the maturation of a monolayer endothelium. The formation of a monolayer from the interfacially polymerized ECs can require that the ECs remodel the fibrinogen and migrate to form cell-cell junctions characteristic of native endothelium. The inventors hypothesized that the requisite 3D migration would occur rapidly enough to give rise to an endothelial monolayer over a roughly weeklong time course.

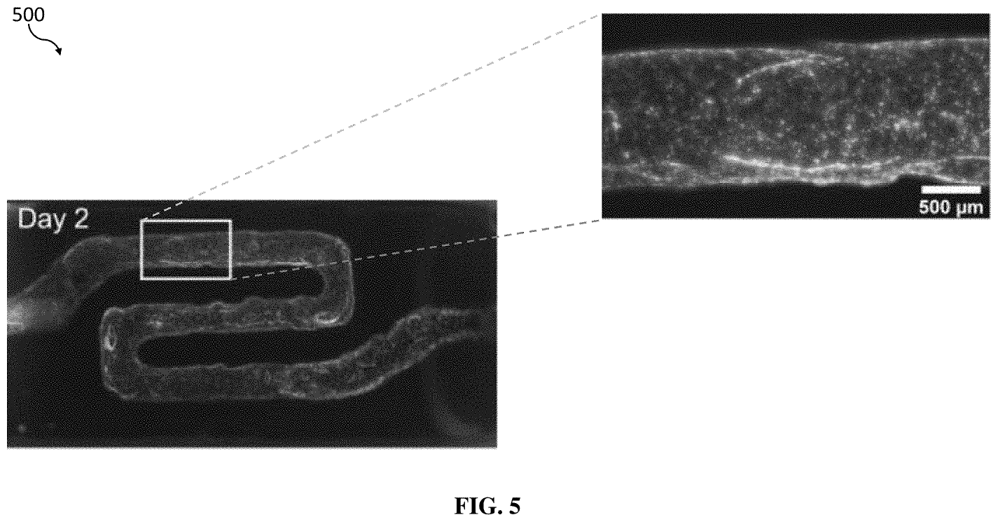

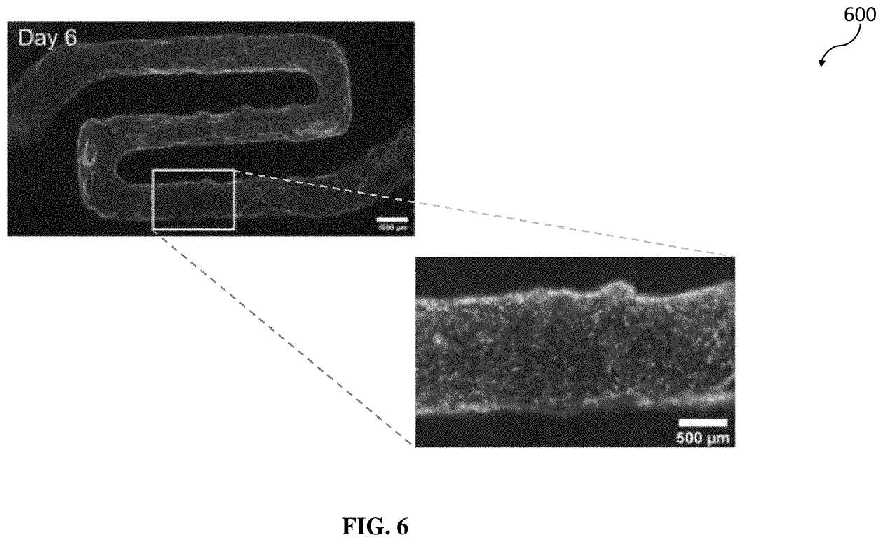

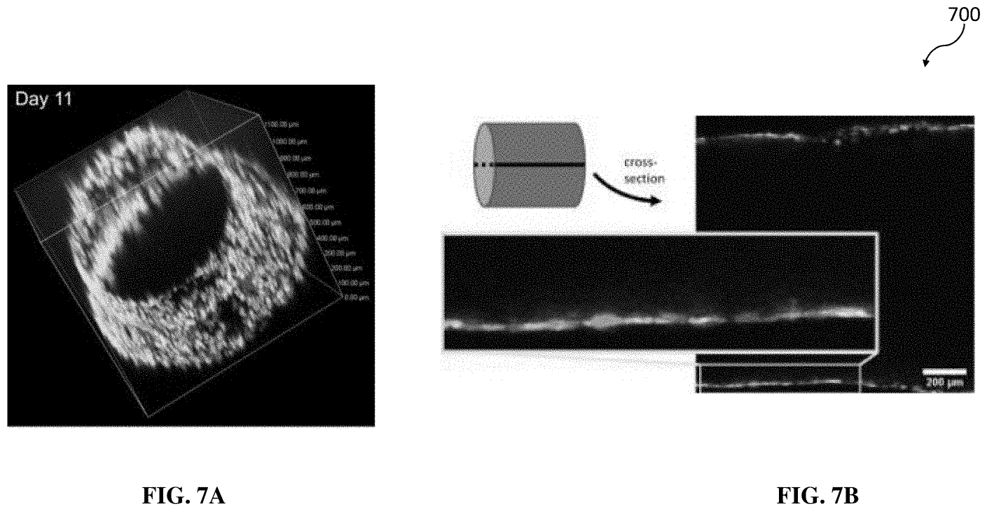

Initially after interfacial polymerization following the seeding of a population of human umbilical vein ECs (HUVECs; mixture of red fluorescent protein (RFP) and green fluorescent protein (GFP) labeled) in a perfused serpentine network, the HUVECs have a rounded morphology reflective of their suspended state within the pre-hydrogel. The mixed population of GFP- and RFP-labeled HUVECs were used to visualize more clearly the morphology of individual cells. Over several days (e.g., a week), i.e., at day 0 (FIG. 4), day 2 (FIG. 5), and day 6 (FIG. 6), and day 11 (FIGS. 7A-7B), the HUVECs adopt a spread morphology and begin to migrate from the rounded morphology of initial interfacial seeding towards one another to form a putative monolayer. Confocal cross-sections through a seeded channel illustrate tightly packed HUVECs along the channel wall in an apparent monolayer. That is, confocal imaging after a week of perfusion, i.e., day 11 as shown in FIGS. 7A-7B, demonstrated uniform coverage around the channel and showed that the ECs had formed a putative compacted monolayer along the channel surface with close interdigitation of adjacent individual cells. In FIGS. 4-7B, primary gel (i.e., the target material) is 20 mg/mL fibrin, and interfacially polymerized gel (i.e., the polymerizable material) is 10 mg/mL fibrin.

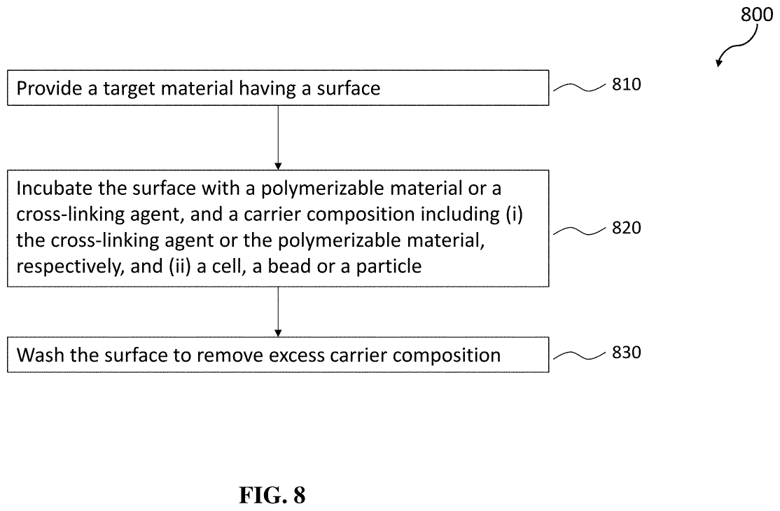

FIG. 8 is a flowchart for a method of coating a target material using a cross-linking agent to catalyze polymerization of a carrier composition, according to various embodiments. As illustrated, the method 800 includes a number of enumerated steps, but aspects of the method 800 may include additional steps before, after, and in between the enumerated steps. In some embodiments, one or more of the enumerated steps may be omitted or performed in a different order.

At block 810, a target material having a surface is provided.

At block 820, the surface is incubated with a polymerizable material or a cross-linking agent, and a carrier composition including (i) the cross-linking agent or the polymerizable material, respectively, and (ii) a cell, a bead or a particle. That is, in some embodiments, the surface is incubated with a polymerizable material, and a carrier composition including a cross-linking agent, and a cell, a bead or a particle. In some embodiments, the surface is incubated with a cross-linking agent and a carrier composition including a polymerizable material, and a cell, a bead or a particle.

At block 830, the surface is washed to remove excess carrier composition. In various embodiments, the cell, the bead or the particle is immobilized on or in an interfacial layer of the material polymerized on the surface.

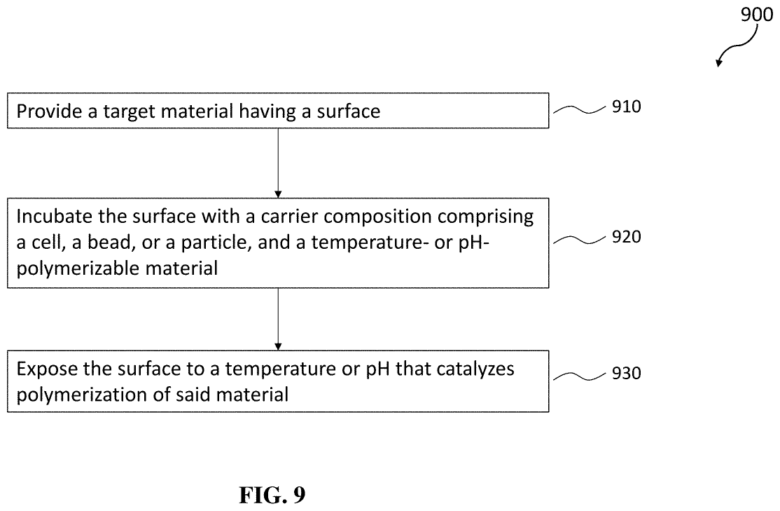

FIG. 9 is a flowchart for a method of coating a target material using a cross-linking agent to catalyze polymerization of a carrier composition, according to various embodiments. As illustrated, the method 900 includes a number of enumerated steps, but aspects of the method 900 may include additional steps before, after, and in between the enumerated steps. In some embodiments, one or more of the enumerated steps may be omitted or performed in a different order.

At block 910, a target material having a surface is provided.

At block 920, the surface is incubated with a carrier composition comprising a cell, a bead, or a particle, and a temperature- or pH-polymerizable material.

At block 930, the surface is exposed to a temperature or pH that catalyzes polymerization of said material. In various embodiments, the cell, the bead or the particle is immobilized on or in an interfacial layer of the material polymerized on the surface.

### Recitation of Various Embodiments of the Present Disclosure

Embodiment 1: A method of coating a target material, the method comprising: providing a target material having a surface; incubating the surface with a polymerizable material or a cross-linking agent, and a carrier composition including (i) the cross-linking agent or the polymerizable material, respectively, and (ii) a cell, a bead or a particle; and washing the surface to remove excess carrier composition, wherein, the cell, the bead or the particle is immobilized on or in an interfacial layer of the material polymerized on the surface.

Embodiment 2: The method of embodiment 1, further comprising washing the surface to remove unbound cross-linking agent after the surface is incubated with the cross-linking agent or the carrier composition including the cross-linking agent.

Embodiment 3: The method of embodiment 1 or 2, wherein the carrier composition is a liquid or a gas.

Embodiment 4: The method of any of embodiments 1-3, wherein a duration of the incubating is modulated to control a thickness of the interfacial layer.

Embodiment 5: The method of any of embodiments 1-4, wherein the interfacial layer is orthogonal or conformal to said surface.

Embodiment 6: The method of any of embodiments 1-5, wherein the cell is a lumenal cell including a fibroblast, a pericyte, an endothelial cell, an epithelial cell, or a smooth muscle cell.

Embodiment 7: The method of any of embodiments 1-6, wherein the bead or the particle includes or is linked to a detectable label, a sensor, or a therapeutic agent.

Embodiment 8: The method of any of embodiments 1-7, wherein the bead is a magnetic bead, a polymeric bead, a PEG microsphere, or a gelatin microsphere.

Embodiment 9: The method of any of embodiments 1-8, wherein the particle is a nanoparticle or a liposome.

Embodiment 10: The method of any of embodiments 1-9, wherein the cell includes a plurality of cells that are of same cell type.

Embodiment 11: The method of any of embodiments 1-9, wherein the cell includes a plurality of cells that are of different cell types.

Embodiment 12: The method of embodiment 11, wherein the different cell types include endothelial cells and stromal cells.

Embodiment 13: The method of embodiment 12, wherein the stromal cells include mesenchymal stem cells or pericytes.

Embodiment 14: The method of any of embodiments 1-13, wherein the surface is located adjacent to either of smooth muscle cells or endothelial cells of concentric layers of the smooth muscle cells and the endothelial cells.

Embodiment 15: The method of embodiment 14, wherein the surface is located in between the concentric layers of the smooth muscle cells and the endothelial cells.

Embodiment 16: The method of any of embodiments 1-15, the cell produces or secretes a factor that promotes or inhibits immobilization.

Embodiment 17: The method of any of embodiments 1-16, wherein the target material is a hydrogel, a biomaterial, or a decellularized tissue or organ.

Embodiment 18: The method of embodiment 17, wherein the biomaterial includes fibrin, gelatin, hyaluronic acid, agarose, alginate, collagen, or decellularized extracellular matrix.

Embodiment 19: The method of embodiment 17, wherein the decellularized tissue or organ includes artery, vein, lymphatic vessel, trachea, esophagus, lung, liver, kidney, pancreas, ureter, bladder, intestines, or urethra.

Embodiment 20: The method of any of embodiments 17-19, wherein the hydrogel is a 3D-printed hydrogel.

Embodiment 21: The method of any of embodiments 17-20, wherein the hydrogel is 3D-printed by stereolithography.

Embodiment 22: The method of any of embodiment 17, wherein the hydrogel is formed by casting around a sacrificial template including a vascular template.

Embodiment 23: The method of any of embodiment 22, wherein the sacrificial template is made of a carbohydrate-based material formed through extrusion or selective laser sintering.

Embodiment 24: The method of any of embodiments 1-23, wherein the surface is an outside of a tube having one or more openings.

Embodiment 25: The method of any of embodiments 1-24, wherein the surface is a cylindrical channel, a hemicylinder, an open void, or a cavity including one or more openings.

Embodiment 26: The method of any of embodiments 1-25, wherein (i) the cross-linking agent is thrombin and the polymerizable material is fibrinogen; (ii) the cross-linking agent is transglutaminase and the polymerizable material is gelatin or gelatin methacrylate; (iii) the cross-linking agent is Ca²⁺ and the polymerizable material is alginate; (iv) the cross-linking agent is ammonium persulfate/TEMED and the polymerizable material is gelatin methacrylate, PEG-diacrylate, collagen methacrylate, silk methacylate, hyaluronic acid methacrylate, chondroitin sulfate methacrylate, elastin methacrylate, cellulose acrylate, dextran methacrylate, heparin methacrylate, NIPAAm methacrylate, chitosan methacrylate, methacrylated decellularized ECM, PEG based peptide conjugates, or combinations thereof; (v) the cross-linking agent is a cysteine-terminated peptide and the polymerizable material is PEG-diacrylate; (vi) the cross-linking agent is lithium acylphosphinate/light and the polymerizable material is gelatin methacrylate, PEG-diacrylate, collagen methacrylate, silk methacylate, hyaluronic acid methacrylate, chondroitin sulfate methacrylate, elastin methacrylate, cellulose acrylate, dextran methacrylate, heparin methacrylate, NIPAAm methacrylate, chitosan methacrylate, methacrylated decellularized ECM, PEG based peptide conjugates, or combinations thereof; or (vii) the cross-linking agent is peroxidase and the polymerizable material is silk fibroin.

Embodiment 27: The method of any of embodiments 1-25, wherein the cross-linking agent and the polymerizable material are a click-chemistry pair.

Embodiment 28: The method of embodiment 27, wherein the click-chemistry pair includes PEG-dithiol, PEG8-norbornene, thiolated gelatin, thiolated chitosan, thiolated silk, thiolated decellularized ECM, or combinations thereof.

Embodiment 29: The method of any of embodiment 1-25, wherein the cross-linking agent is activated by light.

Embodiment 30: The method of embodiment 29, wherein the carrier composition exhibits an index of refraction greater than the target material.

Embodiment 31: The method of embodiment 29, further comprising employing a waveguide to limit exposure of the cross-linking agent to light activation.

Embodiment 32: The method of any of embodiments 1-31, wherein the polymerizable material is polymerized by the cross-linking agent via formation of a covalent or non-covalent bonding.

Embodiment 33: The method of any of embodiments 1-32, wherein the incubating and the washing occur in less than 1 hour, less than 45 mins, less than 30 mins, less than 25, mins, less than 20 mins, less than 15 mins, less than 12 mins, less than 11 mins, less than 10 mins, less than 9 mins, less than 8 mins, less than 7 mins, less than 6 mins, less than 5 mins, less than 4 mins, less than 3 mins, or less than 2 mins, but occur for at least 1 min.

Embodiment 34: The method of any of embodiments 1-33, wherein the incubating the surface includes a first incubation of the surface with the polymerizable material or the cross-linking agent and a second incubation of the surface with the carrier composition; and the surface is immobilized during the first incubation and the washing the surface.

Embodiment 35: The method of embodiment 34, wherein the surface is immobilized during the second incubation.

Embodiment 36: The method of any of embodiments 1-35, further comprising incubating the immobilized cell under conditions supporting cell proliferation and/or cell migration.

Embodiment 37: The method of embodiment 36, wherein the incubating the cell occurs for 1-12 weeks, 1-10 weeks, 1-8 weeks, 1-6 weeks, 1-4 weeks, 1-3 weeks, 1-2 weeks, 1-14 days, 1-10 days, 1-5 days, 2-10 days, 2-6 days, 2-4 days, 5-12 days, 5-10 days, or 12-24 hours.

Embodiment 38: The method of any of embodiments 1-37, wherein a ratio of a thickness of the surface to a thickness of the interfacial layer is between about 100:1 to about 10,000:1.

Embodiment 39: The method of any of embodiments 1-37, wherein a thickness of the interfacial layer is between about 10-1000 microns.

Embodiment 40: The method of any of embodiments 1-39, wherein: the incubating the surface includes a first incubation of the surface with the polymerizable material or the cross-linking agent and a second incubation of the surface with the carrier composition; and the second incubation lasts about 5 seconds to about 10 mins, about 5 seconds to about 30 seconds, about 5 second to about 1 min, about 1 min to about 3 mins, about 1 min to about 5 mins, or about 1 min to about 10 mins.

Embodiment 41: The surface coated with the cell according to the method of embodiments 1-40.

Embodiment 42: The cell coated surface of embodiment 41, wherein the surface is a flat or planar surface.

Embodiment 43: The cell coated surface of embodiment 41, wherein the surface is a non-flat or non-planar surface.

Embodiment 44: The cell coated surface of embodiment 41, wherein the surface is a macroporous structure including a sponge, a woven material, foam, a rectilinear grid, a triangular grid, a gyroid, a honeycomb, or an octet.

Embodiment 45: The cell coated surface of embodiment 41, wherein the surface is located in an implantable device, an artificially engineered or decellularized tissue, an artificially engineered or decelluarized tissue organ, an artificial engineered ductal network, or a cell culture device.

Embodiment 46: The cell coated surface of embodiment 45, wherein the artificial engineered ductal network includes a pancreas, a kidney, or a lung.

Embodiment 47: The cell coated surface of embodiment 45, wherein the artificially engineered or decelluarized tissue organ includes a vasculature network or a lymphatic network.

Embodiment 48: A cell coated lumenal surface comprising: a lumenal surface; an interfacial layer of polymerized material disposed on said lumenal surface; and a cell embedded in said interfacial layer.

Embodiment 49: A method of coating a target material, the method comprising: providing a target material having a surface; incubating the surface with a carrier composition comprising a cell, a bead, or a particle, and a temperature- or pH-polymerizable material; and exposing the surface to a temperature or pH that catalyzes polymerization of said material, wherein: the cell, the bead or the particle is immobilized on or in an interfacial layer of the material polymerized on the surface.

Embodiment 50: The method of embodiment 49, further comprising washing the surface to remove unbound carrier composition.

Embodiment 51: The method of embodiment 49, further comprising washing the surface to remove excess carrier composition.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

A reference to an element in the singular is not intended to mean “one and only one” unless specifically stated, but rather “one or more.” As used herein, the term “about” used with respect to numerical values or parameters or characteristics that can be expressed as numerical values means within ten percent of the numerical values. For example, “about 50” means a value in the range from 45 to 55, inclusive.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method of coating a target material, the method comprising:
providing a target material having a surface;
incubating the surface with a polymerizable material or a cross-linking agent, and a carrier composition including (i) the cross-linking agent or the polymerizable material, respectively, and (ii) a cell, a bead or a particle; and
washing the surface to remove excess carrier composition, wherein,
the cell, the bead or the particle is immobilized on or in an interfacial layer of the material polymerized on the surface.

2. The manifold of claim 1, further comprising washing the surface to remove unbound cross-linking agent after the surface is incubated with the cross-linking agent or the carrier composition including the cross-linking agent.

3. The method of claim 1, wherein a duration of the incubating is modulated to control a thickness of the interfacial layer.

4. The method of claim 1, wherein the cell is a lumenal cell including a fibroblast, a pericyte, an endothelial cell, an epithelial cell, or a smooth muscle cell.

5. The method of claim 1, wherein the bead or the particle includes or is linked to a detectable label, a sensor, or a therapeutic agent.

6. The method of claim 1, wherein the surface is located in between the concentric layers of smooth muscle cells and endothelial cells.

7. The method of claim 1, wherein the target material is a hydrogel, a biomaterial, or a decellularized tissue or organ.

8. The method of claim 7, wherein the hydrogel is a 3D-printed hydrogel.

9. The method of claim 7, wherein the hydrogel is formed by casting around a sacrificial template including a vascular template.

10. The method of claim 1, wherein (i) the cross-linking agent is thrombin and the polymerizable material is fibrinogen; (ii) the cross-linking agent is transglutaminase and the polymerizable material is gelatin or gelatin methacrylate; (iii) the cross-linking agent is Ca²⁺ and the polymerizable material is alginate; (iv) the cross-linking agent is ammonium persulfate/TEMED and the polymerizable material is gelatin methacrylate, PEG-diacrylate, collagen methacrylate, silk methacylate, hyaluronic acid methacrylate, chondroitin sulfate methacrylate, elastin methacrylate, cellulose acrylate, dextran methacrylate, heparin methacrylate, NIPAAm methacrylate, chitosan methacrylate, methacrylated decellularized ECM, PEG based peptide conjugates, or combinations thereof; (v) the cross-linking agent is a cysteine-terminated peptide and the polymerizable material is PEG-diacrylate; (vi) the cross-linking agent is lithium acylphosphinate/light and the polymerizable material is gelatin methacrylate, PEG-diacrylate, collagen methacrylate, silk methacylate, hyaluronic acid methacrylate, chondroitin sulfate methacrylate, elastin methacrylate, cellulose acrylate, dextran methacrylate, heparin methacrylate, NIPAAm methacrylate, chitosan methacrylate, methacrylated decellularized ECM, PEG based peptide conjugates, or combinations thereof; or (vii) the cross-linking agent is peroxidase and the polymerizable material is silk fibroin.

11. The method of claim 1, wherein the cross-linking agent and the polymerizable material are a click-chemistry pair.

12. The method of claim 1, wherein the cross-linking agent is activated by light.

13. The method of claim 13, wherein the carrier composition exhibits an index of refraction greater than the target material.

14. The method of claim 1, wherein a ratio of a thickness of the surface to a thickness of the interfacial layer is between about 100:1 to about 10,000:1.

15. The surface coated with the cell according to the method of claim 1.

16. The cell coated surface of claim 15, wherein the surface is a macroporous structure including a sponge, a woven material, foam, a rectilinear grid, a triangular grid, a gyroid, a honeycomb, or an octet.

17. The cell coated surface of claim 15, wherein the surface is located in an implantable device, an artificially engineered or decellularized tissue, an artificially engineered or decelluarized tissue organ, an artificial engineered ductal network, or a cell culture device.

18. The cell coated surface of claim 15, wherein the artificially engineered or decelluarized tissue organ includes a vasculature network or a lymphatic network.

19. A cell coated lumenal surface comprising:
a lumenal surface;
an interfacial layer of polymerized material disposed on said lumenal surface; and
a cell embedded in said interfacial layer.

20. A method of coating a target material, the method comprising:
providing a target material having a surface;
incubating the surface with a carrier composition comprising a cell, a bead, or a particle, and a temperature- or pH-polymerizable material; and
exposing the surface to a temperature or pH that catalyzes polymerization of said material, wherein:
the cell, the bead or the particle is immobilized on or in an interfacial layer of the material polymerized on the surface.
